SEQUENCE LISTING (49 sequences)

SEQ ID NO: 1 - protein, 8 aa, Artificial Sequence
  Substrate
DEGWALQH

SEQ ID NO: 2 - protein, 8 aa, Artificial Sequence
  Substrate
VGKWSYRM

SEQ ID NO: 3 - protein, 8 aa, Artificial Sequence
  Substrate
NMKWTRVL

SEQ ID NO: 4 - protein, 8 aa, Artificial Sequence
  Substrate
PWTWYGVK

SEQ ID NO: 5 - protein, 8 aa, Artificial Sequence
  Substrate
FGIFYLNG

SEQ ID NO: 6 - protein, 8 aa, Artificial Sequence
  Substrate
HMIALYWG

SEQ ID NO: 7 - protein, 8 aa, Artificial Sequence
  Substrate
IKILMFYW

SEQ ID NO: 8 - protein, 7 aa, Artificial Sequence
  Substrate
GLYFRYE

SEQ ID NO: 9 - protein, 7 aa, Artificial Sequence
  Substrate
AGFSLPA

SEQ ID NO: 10 - protein, 368 aa, Homo sapiens
LHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 11 - protein, 396 aa, Homo sapiens
MKTLLLLLLVLLELGEAQGSLHRVPLRRHPSLKKKLRARSQLSEFWKSHNLDMIQFTESCSMDQSAKEPLINYLDMEYFGTISIGSPPQNFTVIFDTGSSNLWVPSVYCTSPACKTHSRFQPSQSSTYSQPGQSFSIQYGTGSLSGIIGADQVSVEGLTVVGQQFGESVTEPGQTFVDAEFDGILGLGYPSLAVGGVTPVFDNMMAQNLVDLPMFSVYMSSNPEGGAGSELIFGGYDHSHFSGSLNWVPVTKQAYWQIALDNIQVGGTVMFCSEGCQAIVDTGTSLITGPSDKIKQLQNAIGAAPVDGEYAVECANLNVMPDVTFTINGVPYTLSPTAYTLLDFVDGMQFCSSGFQGLDIHPPAGPLWILGDVFIRQFYSVFDRGNNRVGLAPAVP

SEQ ID NO: 12 - protein, 388 aa, Homo sapiens
MKWMVVVLVCLQLLEAAVVKVPLKKFKSIRETMKEKGLLGEFLRTHKYDPAWKYRFGDLSVTYEPMAYMDAAYFGEISIGTPPQNFLVLFDTGSSNLWVPSVYCQSQACTSHSRFNPSESSTYSTNGQTFSLQYGSGSLTGFFGYDTLTVQSIQVPNQEFGLSENEPGTNFVYAQFDGIMGLAYPALSVDEATTAMQGMVQEGALTSPVFSVYLSNQQGSSGGAVVFGGVDSSLYTGQIYWAPVTQELYWQIGIEEFLIGGQASGWCSEGCQAIVDTGTSLLTVPQQYMSALLQATGAQEDEYGQFLVNCNSIQNLPSLTFIINGVEFPLPPSSYILSNNGYCTVGVEPTYLSSQNGQPLWILGDVFLRSYYSVYDLGNNRVGFATAA

SEQ ID NO: 13 - protein, 8 aa, Artificial Sequence
  Substrate
  MISC_FEATURE (1)..(1): Xaa is Asp
  MISC_FEATURE (2)..(2): Xaa is Glu, Phe, Trp or Tyr
  MISC_FEATURE (3)..(3): Xaa is Gly, Asp, Met or Arg
  MISC_FEATURE (4)..(4): Xaa is Tyr, Phe, Trp, Leu or Met
  MISC_FEATURE (5)..(5): Xaa is Tyr, Met, Leu, Ala, Phe or Ser
  MISC_FEATURE (6)..(6): Xaa is Leu, Thr or Arg
  MISC_FEATURE (7)..(7): Xaa is Gln or Tyr
  MISC_FEATURE (8)..(8): Xaa is His
XXXXXXXX

SEQ ID NO: 14 - protein, 4 aa, Artificial Sequence
  Substrate
  MISC_FEATURE (1)..(1): Xaa is Gly, Lys, Thr, Asp, Met, Ile, Leu, Ser or Arg, or a modified amino acid residue thereof
  MISC_FEATURE (2)..(2): Xaa is Tyr, Phe, Ala, Trp, Leu, Tyr, Nle or Met, or a modified amino acid residue thereof
  MISC_FEATURE (3)..(3): Xaa is Tyr, Met, Leu, Ala, Phe, Thr, Pro or Ser, or a modified amino acid residue thereof
  MISC_FEATURE (4)..(4): Xaa is Leu, Tyr, Gly, Ala, Thr or Arg, or a modified amino acid residue thereof
XXXX

SEQ ID NO: 15 - protein, 5 aa, Artificial Sequence
  Cleavage site
  MISC_FEATURE (2)..(2): Xaa is Nle
RXLFV

SEQ ID NO: 16 - protein, 5 aa, Artificial Sequence
  Cleavage site
HPFWI

SEQ ID NO: 17 - protein, 5 aa, Artificial Sequence
  Cleavage site
  MISC_FEATURE (3)..(3): Xaa is Nle
  MISC_FEATURE (5)..(5): Xaa is Nle
YAXYX

SEQ ID NO: 18 - protein, 5 aa, Artificial Sequence
  Cleavage site
KFYVL

SEQ ID NO: 19 - protein, 5 aa, Artificial Sequence
  Cleavage site
QYWIW

SEQ ID NO: 20 - protein, 5 aa, Artificial Sequence
  Cleavage site
FWEAF

SEQ ID NO: 21 - protein, 5 aa, Artificial Sequence
  Cleavage site
IIDTY

SEQ ID NO: 22 - protein, 5 aa, Artificial Sequence
  Cleavage site
AVQLS

SEQ ID NO: 23 - protein, 5 aa, Artificial Sequence
  Cleavage site
  MISC_FEATURE (4)..(4): Xaa is Nle
STAXT

SEQ ID NO: 24 - protein, 5 aa, Artificial Sequence
  Cleavage site
EENSA

SEQ ID NO: 25 - protein, 5 aa, Artificial Sequence
  Cleavage site
TLRER

SEQ ID NO: 26 - protein, 5 aa, Artificial Sequence
  Cleavage site
VSKNQ

SEQ ID NO: 27 - protein, 5 aa, Artificial Sequence
  Cleavage site
NGSDH

SEQ ID NO: 28 - protein, 5 aa, Artificial Sequence
  Cleavage site
  MISC_FEATURE (1)..(1): Xaa is Nle
XQHHN

SEQ ID NO: 29 - protein, 5 aa, Artificial Sequence
  Cleavage site
WKVRK

SEQ ID NO: 30 - protein, 5 aa, Artificial Sequence
  Cleavage site
LNIQE

SEQ ID NO: 31 - protein, 5 aa, Artificial Sequence
  Cleavage site
GDTGD

SEQ ID NO: 32 - protein, 5 aa, Artificial Sequence
  Cleavage site
DRGKG

SEQ ID NO: 33 - protein, 5 aa, Artificial Sequence
  Cleavage site
PHPPP

SEQ ID NO: 34 - protein, 7 aa, Artificial Sequence
  Substrate
  MISC_FEATURE (1)..(1): Xaa is Ala, Gly, His or Nle
  MISC_FEATURE (2)..(2): Xaa is Asp or Gly
  MISC_FEATURE (3)..(3): Xaa is Leu, Nle, Asp, Gln, Tyr, Phe, Trp, Pro or Gly
  MISC_FEATURE (4)..(4): Xaa is Tyr, Phe, Ile, Ala, Val, Gly, Arg or Pro
  MISC_FEATURE (5)..(5): Xaa is Asp or Pro
  MISC_FEATURE (6)..(6): Xaa is Tyr, Gly or Gln
  MISC_FEATURE (7)..(7): Xaa is Lys, Gly, Arg, Trp or Gln
XXXXXXX

SEQ ID NO: 35 - protein, 7 aa, Artificial Sequence
  Substrate
  MISC_FEATURE (4)..(4): Xaa id Nle
EAWXTFI

SEQ ID NO: 36 - protein, 7 aa, Artificial Sequence
  Substrate
  MISC_FEATURE (1)..(1): Xaa is lysine-fluorophore
  MISC_FEATURE (4)..(4): Xaa is Nle
XAWXTFI

SEQ ID NO: 37 - protein, 7 aa, Artificial Sequence
  Substrate
  MISC_FEATURE (2)..(2): Xaa is lysine-fluorophore
  MISC_FEATURE (4)..(4): Xaa is Nle
EXWXTFI

SEQ ID NO: 38 - protein, 7 aa, Artificial Sequence
  Substrate
  MISC_FEATURE (3)..(3): Xaa is lysine-fluorophore
  MISC_FEATURE (4)..(4): Xaa is Nle
EAXXTFI

SEQ ID NO: 39 - protein, 7 aa, Artificial Sequence
  Substrate
INDFLVR

SEQ ID NO: 40 - protein, 7 aa, Artificial Sequence
  Substrate
GLYFRYE

SEQ ID NO: 41 - protein, 7 aa, Artificial Sequence
  Substrate
  MISC_FEATURE (1)..(1): Xaa is lysine-fluorophore
XLYFRYE

SEQ ID NO: 42 - protein, 7 aa, Artificial Sequence
  Substrate
  MISC_FEATURE (2)..(2): Xaa is lysine-fluorophore
GXYFRYE

SEQ ID NO: 43 - protein, 7 aa, Artificial Sequence
  Substrate
  MISC_FEATURE (3)..(3): Xaa is lysine-fluorophore
GLXFRYE

SEQ ID NO: 44 - protein, 7 aa, Artificial Sequence
  Substrate
  MISC_FEATURE (3)..(3): Xaa is lysine-fluorophore
INXFLVR

SEQ ID NO: 45 - protein, 7 aa, Artificial Sequence
  Substrate
  MISC_FEATURE (1)..(1): Xaa is lysine-fluorophore
XNDFLVR

SEQ ID NO: 46 - protein, 7 aa, Artificial Sequence
  Substrate
  MISC_FEATURE (2)..(2): Xaa is lysine-fluorophore
IXDFLVR

SEQ ID NO: 47 - skipped

SEQ ID NO: 48 - protein, 8 aa, Artificial Sequence
  Substrate
  MISC_FEATURE (1)..(1): Xaa is Asp, Ala, Val, Asn, Pro, Phe, His, Ile, Glu, Met, Trp, Lys or Gly, or a modified amino acid residue thereof
  MISC_FEATURE (2)..(2): Xaa is Glu, Phe, Gly, Asn, Met, Lys, Ala, Lys, Leu or Trp, or a modified amino acid residue thereof
  MISC_FEATURE (3)..(3): Xaa is Gly, Phe, Lys, Asp, Met, Thr, Ile, Asn, Tyr, Trp, Leu or Arg, or a modified amino acid residue thereof
  MISC_FEATURE (4)..(4): Xaa is Tyr, Ala, Ser, Phe, Asn, Trp, Leu, Thr or Met, or a modified amino acid residue thereof
  MISC_FEATURE (5)..(5): Xaa is Tyr, Met, Asn, Leu, Ala, Phe or Ser, or a modified amino acid residue thereof
  MISC_FEATURE (6)..(6): Xaa is Leu, Thr or Arg, or a modified amino acid residue thereof
  MISC_FEATURE (7)..(7): Xaa is Gln or Tyr, or a modified amino acid residue thereof
  MISC_FEATURE (8)..(8): Xaa is His, Met, Leu, Lys, Asn, Gly or Trp, or a modified amino acid residue thereof
XXXXXXXX

SEQ ID NO: 49 - protein, 7 aa, Artificial Sequence
  Substrate
  MISC_FEATURE (1)..(1): Xaa is Asp, Ala, Val, Asn, Pro, Phe, His, Ile, Glu, Met, Trp, Lys or Gly, or a modified amino acid residue thereof
  MISC_FEATURE (2)..(2): Xaa is Glu, Phe, Gly, Asn, Met, Lys, Ala, Lys, Leu or Trp, or a modified amino acid residue thereof
  MISC_FEATURE (3)..(3): Xaa is Gly, Phe, Lys, Asp, Asn, Met, Thr, Ile, Tyr, Trp, Leu or Arg, or a modified amino acid residue thereof
  MISC_FEATURE (4)..(4): Xaa is Tyr, Ala, Ser, Phe, Trp, Asn, Leu, Thr or Met, or a modified amino acid residue thereof
  MISC_FEATURE (5)..(5): Xaa is Tyr, Met, Asn, Leu, Ala, Phe or Ser, or a modified amino acid residue thereof
  MISC_FEATURE (6)..(6): Xaa is Leu, Thr or Arg, or a modified amino acid residue thereof
  MISC_FEATURE (7)..(7): Xaa is Gln or Tyr, or a modified amino acid residue thereof
XXXXXXX

(19) United States
(12) Patent Application Publication
(10) Pub. No.: US 2022/0381784 A1
(43) Pub. Date: Dec. 1, 2022

(54) SYSTEMS COMPRISING SUBSTRATES AND METHODS OF USING THE SAME FOR DETECTION OF PANCREATIC CANCERS

(71) Applicant: THE REGENTS OF THE UNIVERSITY OF CALIFORNIA, Oakland, CA (US)

(72) Inventors: Charles S. CRAIK, San Francisco, CA (US); Sam L. IVRY, San Francisco, CA (US)

(21) Appl. No.: 17/617,285

(22) PCT Filed: Jun. 8, 2020
(86) PCT No.: PCT/US2020/036702

§ 371 Date: Dec. 7, 2021

Related U.S. Application Data
(60) Provisional application No. 62/858,970, filed on Jun. 7, 2019.

(51) Int. Cl. G01N 33/574; C12Q 1/37

(52) U.S. Cl. CPC G01N 33/57438; C12Q 1/37; G01N 2333/96411

(57) ABSTRACT

Diagnostic platforms for the detection of mucinous and high grade cysts in the pancreas of subjects are provided. The disclosure provides a system comprising enzyme specific substrate, that are specific for detection and/or quantification of serine protease TPP-1, the value of which corresponding to a determination of whether a mucinous cyst in the pancreas is malignant or benign. The disclosure also provides for a two-step method, in which a sample from a pancreatic cyst is determined mucinous by detection and/or quantification of aspartyl protease expression, and then the same cyst is determined high grade dysplasia or malignant by detection and/or quantification of serine protease expression.

### CROSS-REFERENCE TO RELATED APPLICATIONS

This application claims priority to U.S. Provisional Application No. 62/858,970 filed on Jun. 7, 2019, which is incorporated by reference in its entirety

### STATEMENT REGARDING FEDERALLY SPONSORED RESEARCH

This invention was made with government support under grant numbers R21CA186077 and UL1TR000004, T32GM008155 awarded by the National Institutes of Health. The government has certain rights in this invention.

### TECHNOLOGY FIELD

Diagnostic platforms for the detection of mucinous and high grade cysts in the pancreas of subjects are provided. The disclosure provides a system comprising enzyme specific substrate, that are specific for detection and/or quantification of serine protease TPP-1, the value of which corresponding to a determination of whether a mucinous cyst in the pancreas is malignant or benign. The disclosure also provides for a two-step method, in which a sample from a pancreatic cyst is determined mucinous by detection and/or quantification of aspartyl protease expression, and then the same cyst is determined high grade dysplasia or malignant by detection and/or quantification of serine protease expression.

### BACKGROUND

Pancreatic cysts are incidentally detected in 13%-45% of patients being evaluated by abdominal MRI (Lee et al. 2010; Moris et al. 2016). Clinical management is confounded by unreliable risk stratification of the malignant potential of pancreatic cysts. Mucinous cysts, which include intraductal papillary mucinous neoplasms (IPMNs) and mucinous cystic neoplasms (MCNs), are precursor lesions to pancreatic cancer and should be resected if they harbor high-grade dysplasia or invasive cancer (HGD/IC). However, a significant portion of mucinous cysts only contain low-grade dysplasia (LGD). These lesions are generally considered benign and it is currently recommended that they be followed through surveillance for malignant progression. Serous cystadenomas (SCAs) and pancreatic pseudocysts, which are both types of nonmucinous cysts, are also common and present minimal risk to patients if they remain asymptomatic. Unfortunately, it remains challenging to preoperatively classify the cyst type and degree of histological atypia to determine if surgical intervention is warranted.

Current management guidelines for pancreatic cysts are largely based on clinical and radiographic features (Tanaka et al., 2012). These guidelines demonstrate variable sensitivity and unsatisfactory specificity for diagnosing cysts with HGD/IC (Scheiman et al. 2015). In an effort to improve diagnostic accuracy, cyst fluid is now routinely collected by endoscopic ultrasound with fine needle aspiration (EUS-FNA) and subjected to analysis. Evaluation of the tumor marker, carcinoembryonic antigen (CEA), is 60%-75% sensitive and 84%-93% specific for differentiating nonmucinous from mucinous cysts (Brugge et al., 2004; Ivry et al., 2017; Park et al., 2011). However, CEA levels are unable to distinguish mucinous cysts with LGD from those with HGD/IC. Cytological assessment of cyst fluid collected by EUS-FNA is also commonly performed. Although it is highly specific for identifying mucinous cysts with HGD/IC, it suffers from a sensitivity of only 30%-50%, which limits its usefulness (Maker et al. 2008; Waaij et al. 2005).

The limitations of clinicoradiographic and the cyst fluid diagnostics have spurred significant interest in identifying novel molecular biomarkers. Several cyst fluid biomarkers have shown promise for differentiating mucinous from nonmucinous cysts and for determining which mucinous cysts harbor HGD/IC. Examples of these include microRNA (Matthaei et al., 2012; Wang et al., 2015), mucins (Cao et al., 2013; Sinha et al., 2016), glucose levels (Can et al., 2017; Zikos et al., 2015), DNA methylation (Hata et al., 2017), telomerase activity (Hata et al., 2016), and an array of DNA mutations (Singhi et al., 2017; Springer et al., 2015; Wu et al., 2011; Wu et al., 2011). In a recent study, we used a global protease activity profiling technology to identify two aspartyl proteases, gastricsin and cathepsin E, as promising biomarkers for differentiating mucinous from nonmucinous cysts (Ivry et al., 2017). Analysis of gastricsin activity using a simple, fluorescence-based assay was 95% accurate for classifying mucinous cysts. Immunohistochemical analysis revealed that gastricsin expression in mucinous cysts was primarily associated with regions of LGD; however, activity levels were unable to differentiate LGD from HGD/IC.

In the present study, we identify increased aminopeptidase activity in fluid from mucinous cysts and determine that the lysosomal protease TPP1 is primarily responsible for this activity. Using both a highly sensitive, targeted proteomics workflow and a simple, fluorescence-based assay, we demonstrate that TPP1 levels are significantly increased in mucinous cysts relative to nonmucinous cysts. Interestingly, TPP1 activity is primarily associated with HGD/IC, which is a critical factor in determining if a cyst should be surgically resected.

### SUMMARY OF EMBODIMENTS

The present disclosure relates to a method of diagnosing a subject with a benign, pre-malignant, or malignant growth of the pancreas. In one aspect, the method comprises detecting the presence, absence, and/or quantity of at least one tripeptidyl peptidylase or functional fragment thereof in a sample. In one aspect, the method comprises detecting the presence, absence, and/or quantity of at least lysosomal aminopeptidase tripeptidyl peptidase I (TPP1) or functional fragment thereof in a sample. In some embodiments, the method further comprises detecting the presence, absence, and/or quantity of cathepsin E, CEA and/or gastricsin.

The present disclosure also relates to a method of diagnosing a subject with a high-grade dysplasia or invasive cancer (HGD/IC). In one aspect, the method comprises detecting the presence, absence, and/or quantity of at least one tripeptidyl peptidylase or functional fragment thereof in a sample.

The present disclosure also relates to a method of diagnosing a subject with an intraductal papillary mucinous neoplasm (IPMN). In one aspect, the method comprises detecting the presence, absence, and/or quantity of at least one tripeptidyl peptidylase or functional fragment thereof in a sample.

The present disclosure also relates to a method of diagnosing a subject with a mucinous cystic neoplasm (MCN). In one aspect, the method comprises detecting the presence, absence, and/or quantity of at least one tripeptidyl peptidylase or functional fragment thereof in a sample.

The present disclosure also relates to a method of diagnosing a subject with a pseudocyst. In one aspect, the method comprises detecting the presence, absence, and/or quantity of at least one tripeptidyl peptidylase or functional fragment thereof in a sample.

The present disclosure also relates to a method of diagnosing a subject with a mucinous cyst. In one aspect, the method comprises: (a) detecting a presence or quantifying an amount of TPP1, cathepsin E and/or gastricsin and/or functional fragment thereof, in a sample of the subject, by contacting the sample with a substrate specific for TPP1, cathepsin E and/or gastricsin and/or functional fragment thereof; and (b) diagnosing a subject with a mucinous cyst when the presence or quantity of TPP1, cathepsin E and/or gastricsin and/or functional fragment thereof is detected or quantified.

The present disclosure also relates to a method of diagnosing a subject with pancreatic cancer. In one aspect, the method comprises: (a) detecting a presence or quantifying an amount of TPP1 and/or functional fragment thereof, in a sample of the subject, by contacting the sample with a substrate specific for TPP1 and/or functional fragment thereof; and (b) diagnosing a subject with pancreatic cancer when the presence or quantity of TPP1 and/or functional fragment thereof is detected or quantified.

The present disclosure also relates to a method of treating a subject in need thereof diagnosed with or suspected of having pancreatic cancer. In one aspect, the method comprises: (a) contacting a plurality of probes specific for TPP1 and/or functional fragment thereof with a sample; (b) quantifying the amount of TPP1 and/or functional fragment thereof in the sample; (c) calculating one or more scores based upon the presence, absence, or quantity of TPP1 and/or functional fragment thereof; (d) correlating the one or more scores to the presence, absence, or quantity of TPP1 and/or functional fragment thereof, such that if the amount of TPP1 and/or functional fragment thereof is greater than the quantity of TPP1 and/or functional fragment thereof in a control sample, the correlating step comprises diagnosing a subject with pancreatic cancer; and (e) administering to the subject a therapeutically effective amount of treatment for the pancreatic cancer.

### BRIEF DESCRIPTIONS OF THE DRAWINGS

FIG. 1A-1C depicts identification of low pH triamino activity in mucinous cysts through MSP-MS. FIG. 1A: The average number of detected cleavages between each amide bond (BX) within the 228 peptide library when the MSP-MS assay was performed at pH 3.5. A representative 14-mer library peptide is shown below the bar chart with black circles indicating amino acids. Two-way ANOVA was used for determining statistical significance (*P<0.05, **P<0.005, ***P<0.0005, ****P<0.00005). FIG. 1B: Heatmap displaying the number of cleavages at each amide bond (BX) following treatment of a mucinous cyst fluid sample with DMSO, pepstatin, Ala-Ala-Phe-chloromethylketone (AAF-CMK), or a combination. FIG. 1C: Volcano plot comparing the amount of cleavage at the third amide bond for individual library peptides. We detected cleavage of 93 library peptides at this bond. Spectral counts of cleavage products were used for quantification of the fold change (mucinous/nonmucinous) and hypothesis testing. Dotted lines indicate P<0.05 and ±1 log 2(fold change) and blue dots are used for triamino peptide cleavages that met both of these criteria.

FIG. 2A: Average number of detected peptide cleavages during MSP-MS analysis at pH 3.5 and 7.5. FIG. 2B: The average number of detected cleavages between each amide bond when the MSP-MS assay was performed at pH 7.5. FIG. 2C: Heatmap showing the number of detected cleavages at each amide bond following treatment of a mucinous cyst with DMSO or a panel of broad-spectrum protease inhibitors. For all analysis, two-way ANOVA was used for determining statistical significance (**P<0.005).

FIG. 3A-3C depicts that TPP1 abundance is increased in fluid from mucinous cysts. Venn diagrams showing the peptidases (FIG. 2A) and aminopeptidases (FIG. 2B) identified through shotgun proteomic analysis of fluid from mucinous (blue, n=3) and nonmucinous (red, n=3) cysts. (FIG. 2C) PRM analysis TPP1 peptides from nonmucinous and mucinous cyst fluid. Integrated peak areas of the eight most abundant transitions were used to determine the fold change (mucinous/nonmucinous). An unpaired, two-tailed t-test of the log 2(fold change) was used for determining statistical significance.

FIG. 4 depicts PRM analysis of peptides from PGC and CTSE in fluid from nonmucinous (NM, n=3) and mucinous (M, n=3) cysts. Peak areas of the eight most abundant transitions were used to determine the fold change and assess statistical significance using unpaired, two-tailed t-tests.

FIG. 5A-5C depicts development of an internally quenched fluorescent substrate for TPP1 activity analysis in cyst fluid. FIG. 5A: iceLogo depicting the substrate specificity of recombinant TPP1 based on the 202 peptide cleavages detected through MSP-MS analysis. Amino acids that are significantly enriched at specific positions are shown in black (P<0.05), while those that trended towards being enriched are in grey (P<0.20). FIG. 5B: Relative cleavage of a peptide from the MSP-MS library in nonmucinous and mucinous cysts. Arrow indicates that cleavage is occurring after the third amino acid from the N-terminus and the 7 remaining C-terminal amino acids aren't shown. Spectral counts were used for relative quantification and an unpaired, two-tailed t-test was used for statistical testing. FIG. 5C: Cleavage of internally quenched fluorescent substrates by recombinant TPP1. X indicates the position of the lysine-fluorophore.

FIG. 6A: Time dependent cleavage of 3 peptides in the MSP-MS library. Arrow indicates that cleavage is after the third amino acid and the 7 remaining C-terminal amino acids aren't shown. FIG. 6B: Triamino cleavage of two peptides from FIG. 6A by cyst fluid samples. An unpaired, two-tailed t-test was used for statistical testing. FIG. 6C: Cleavage of internally quenched fluorescent substrates by TPP1. The position of the lysine-fluorophore is indicated by X.

FIG. 7A: Kinetic analysis of TPP1 cleavage of our most sensitive internally quenched fluorescent substrate. TPP1 concentration was constant at 2 μmol/L, while substrate concentration ranged from 0.1-50 μmol/L. FIG. 7B: Determination of limit of detection of TPP1 activity using internally quenched fluorescent substrate. Substrate concentration was 20 μmol/L and TPP1 concentration ranged from 95-3000 pg/mL.

FIG. 8A-8D depict Analysis of TPP1 activity in cyst fluid samples. FIG. 8A: Comparison of TPP1 activity levels in nonmucinous and mucinous cysts and ROC curve (FIG. 8B) for differentiating these cyst types. FIG. 8C: TPP1 activity in mucinous cysts of varying degrees of dysplasia and associated ROC curve (FIG. 8D) for distinguishing LGD from HGD/IC. For all statistical testing, an unpaired, two-tailed t-test was used.

FIG. 9 depicts TPP1 activity in different types of cysts. Statistical testing was done with an unpaired, two-tailed t-test.

FIG. 10 depicts a chart depicting the biomarkers tested for determining the difference between a Low Grade Vs. High Grade Dysplasia/Invasive Pancreatic Cysts. (“-”) indicates that the biomarker is not predictive of the conditions. “++” indicates that the biomarker is aligns with the identified category to a degree of above about 80% of the time. “•” indicates that the biomarker is aligns with the identified category to a degree of above about 80% of the time.

### DETAILED DESCRIPTION OF THE EMBODIMENTS

Before the present compositions and methods are described, it is to be understood that this disclosure is not limited to the particular molecules, compositions, methodologies or protocols described, as these may vary. It is also to be understood that the terminology used in the description is for the purpose of describing the particular versions or embodiments only, and is not intended to limit the scope of the present disclosure which will be limited only by the appended claims. It is understood that these embodiments are not limited to the particular methodology, protocols, cell lines, vectors, and reagents described, as these may vary. It also is to be understood that the terminology used herein is for the purpose of describing particular embodiments only, and is not intended to limit the scope of the present embodiments or claims.

Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the preferred methods, devices, and materials are now described. All publications mentioned herein are incorporated by reference. Nothing herein is to be construed as an admission that the disclosure is not entitled to antedate such disclosure by virtue of prior disclosure.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of” “only one of” or “exactly one of” “Consisting essentially of,” when used in the claims, shall have its ordinary meaning as used in the field of patent law.

The term “about” as used herein when referring to a measurable value such as an amount, a temporal duration, and the like, is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value, as such variations are appropriate to perform the disclosed methods.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

As used herein, “substantially equal” means within a range known to be correlated to an abnormal or normal range at a given measured in some embodiments, “substantially equal” means that the associated term is from about +/−10% of where an equal value would be associated with whatever metric is modified by the term. For example, if a control sample is from a diseased patient, substantially equal is, in some embodiments, within an abnormal range+/−10% of the abnormal value. If a control sample is from a patient known not to have the condition being tested, substantially equal is within a normal range for that given metric. In some embodiments, the amount of expression of enzymes detected by any of the methods disclosed herein is from about 1.01 to about 2.00 times the amount of expression of the enzymes disclosed herein in order for the diagnosis of pre-malignant or malignant to be made. In some embodiments, the amount of expression of enzymes detected by any of the methods disclosed herein is from about 1.01 to about 1.50 times the amount of expression of the enzymes disclosed herein in order for the diagnosis of pre-malignant or malignant to be made.

As used herein, the term “subject,” “individual” or “patient,” used interchangeably, means any animal, including mammals, such as mice, rats, other rodents, rabbits, dogs, cats, swine, cattle, sheep, horses, or primates, such as humans. The term “subject” is used, in some embodiments, throughout the specification to describe an animal from which a sample is taken. In some embodiment, the subject is a human. For diagnosis of those conditions which are specific for a specific subject, such as a human being, the term “patient” may be interchangeably used. In some instances in the description of the present invention, the term “patient” will refer to human patients suffering from a particular disease or disorder. In some embodiments, the subject may be a human suspected of having or being identified as at risk to develop a type of cancer more severe or invasive than initially diagnosed. In some embodiments, the subject may be diagnosed as having at resistance to one or a plurality of treatments to treat a disease or disorder afflicting the subject. In some embodiments, the subject is suspected of having or has been diagnosed with stage I, II, III or greater stage of cancer. In some embodiments, the subject may be a human suspected of having or being identified as at risk to a terminal condition or disorder. In some embodiments, the subject may be a mammal which functions as a source of the isolated sample of biopsy or bodily fluid. In some embodiments, the subject may be a non-human animal from which a sample of biopsy or bodily fluid is isolated or provided.

As used herein, the terms “biologically significant” refers to an amount or concentration of enzymatic reaction product or enzyme in a sample whose quantity of binding that is detected and is statistically significant as compared to a control when the amount or concentration is normalized for a control. In some embodiments, the terms is used to describe the amount of serine protease or aspartyl proteases that is present in a sample at a level sufficient to cause a dysfunctional biological effect. In some embodiments, the biologically significant amount of enzyme is the amount sufficient to characterize a sample as comprising a hyperproliferative cell. In some embodiments, the biologically significant amount of enzyme is the amount sufficient to characterize a sample as being a mucinous pancreatic cyst versus a non-mucinous cyst. In some embodiments, the biologically significant amount of enzyme is the amount sufficient to characterize a sample as comprising at least one or a plurality of cells with a high grade dysplasia or invasively cancerous. In some embodiments, the biologically significant amount of enzyme is the amount sufficient to characterize a sample as comprising at least one or a plurality of cells that are malignantly cancerous of the pancreas.

As used herein, the term “kit” refers to a set of components provided in the context of a system for delivering materials or diagnosing a subject with having a pancreatic cyst. Such delivery systems may include, for example, systems that allow for storage, transport, or delivery of various diagnostic or therapeutic reagents (e.g., oligonucleotides, enzymes, extracellular matrix components etc. in appropriate containers) and/or supporting materials (e.g., buffers, media, cells, written instructions for performing the assay etc.) from one location to another. For example, in some embodiments, kits include one or more enclosures (e.g., boxes) containing relevant reaction reagents and/or supporting materials. As used herein, the term “fragmented kit” refers to a diagnostic assay comprising two or more separate containers that each contain a subportion of total kit components. Containers may be delivered to an intended recipient together or separately. For example, a first container may contain a petri dish or polystyrene plate for use in a cell culture assay, while a second container may contain cells, such as control cells. As another example, the kit may comprise a first container comprising a solid support such as a chip or slide with one or a plurality of ligands with affinities to one or a plurality of biomarkers disclosed herein and a second container comprising any one or plurality of reagents necessary for the detection and/or quantification of the amount of biomarkers in a sample. The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contain a sub-portion of total kit components are included in the term “fragmented kit.” In contrast, a “combined kit” refers to a delivery system containing all components in a single container (e.g., in a single box housing each of the desired components). The term “kit” includes both fragmented and combined kits.

As used herein, “cell culture” means growth, maintenance, transfection, or propagation of cells, tissues, or their products. As used herein, “culture medium” refers to any solution capable of sustaining the growth of the targeted cells either in vitro or in vivo, or any solution with which targeted cells or exogenous nucleic acids are mixed before being applied to cells in vitro or to a patient in vivo. In some embodiments, culture medium means solution capable of sustaining the growth of the targeted cells either in vitro.

As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a non-human mammal.

The terms “enzymatically effective amount” means any amount of the probe or substrate disclosed herein that is at a concentration capable of causing an enzymatic reaction to take place in a detectable amount. In some embodiments, any of the disclosed methods comprise a step of contacting a probe with any disclosed in an enzymatically effective amount. In some embodiments, the enzymatically effective amount is the amount of a substrate fused covalently or non-covalently to a probe, the substrate specific for a serine protease or an aspartyl protease. In some embodiments, the enzymatically effective amount is the amount of a substrate fused covalently or non-covalently to a probe, the substrate specific for TPP-1, gastricsin, and/or cathepsin E, or functional fragments or variants thereof.

As used herein, the term “mammal” means any animal in the class Mammalia such as rodent (i.e., a mouse, a rat, or a guinea pig), a monkey, a cat, a dog, a cow, a horse, a pig, or a human. In some embodiments, the mammal is a human. In some embodiments, the mammal refers to any non-human mammal. The present disclosure relates to any of the methods or compositions of matter disclosed herein wherein the sample is taken from a mammal or non-human mammal. The present disclosure relates to any of the methods or compositions of matter disclosed herein wherein the sample is taken from a human or non-human primate.

As used herein, the phrase “in need thereof” means that the animal or mammal has been identified or suspected as having a need for the particular method or treatment. In some embodiments, the identification can be by any means of diagnosis or observation. In any of the methods and treatments described herein, the animal or mammal can be in need thereof. In some embodiments, the animal or mammal is in an environment or will be traveling to an environment in which a particular disorder or condition is prevalent or more likely to occur. In some embodiments, the subject in need thereof is diagnosed of having cancer or diagnosed with a pancreatic cyst. In some embodiments, the subject is diagnosed as having a pancreatic cyst by MRI, CT or PET scan. In some embodiments, a subject in need thereof is one who is suspected of having pancreatic cancer and/or a subject with a high risk of developing pancreatic cancer.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the terms “hyperproliferative cell” means any cell of any subject that exhibits a disorder caused by dysfunction of its growth and/or division cycle. Hyperproliferative cells may be cancerous, benign or malignant. In some embodiments, the hyperproliferative cell is cancerous or precancerous. In some embodiments, the hyperoliferative cell is caused by loss of function and cellular senescence. In some embodiments, the hyperproliferative cell is caused by the overexpression of protein responsible for cellular division.

As used herein, the phrase “therapeutically effective amount” means the amount of active compound or pharmaceutical agent or agent within a pharmaceutical composition that elicits the biological or medicinal response that is being sought in a tissue, system, animal, individual or human by a researcher, veterinarian, human physician or other clinician, such as a pathologist. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

As used herein, the terms “treat,” “treated,” or “treating” can refer to therapeutic treatment and/or prophylactic or preventative measures wherein the object is to prevent or slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment.

As used herein, the terms “diagnose,” “diagnosing,” or variants thereof refer to identifying the nature of a physiological condition, disorder or disease. In some embodiments, diagnosing a subject refers to identifying whether a cyst is benign, pre-malignant, or malignant. In some embodiments, diagnosing refers to distinguishing between a mucinous and non-mucinous cyst. In some embodiments, such a cyst is derived from or in the pancreas of the subject.

As used herein, a “cyst” is a membranous sac or cavity of abnormal character that may contain fluid. Mucinous cysts are a type of cyst that arise from epithelial cells, and have the potential to become malignant. Non-mucinous cysts of the pancreas are considered benign or harmless.

As used herein, “benign” is to be contrasted with “malignant.” The terms “benign” and “malignant” are intended to convey their ordinary meaning. Therefore, “malignant” when modifying a growth is intended to refer to an abnormal growth or hyperproliferative state that is characterized by invasive or potentially invasive growth causing destruction of local tissues and cells, often leading to metastasis and death in the absence of treatment. In contrast, “benign” is intended to refer to an abnormal growth state wherein the growth does not result in the invasion of the local tissue, metastasis, or death. As used herein, “pre-malignant” is intended to refer to an abnormal growth state of a cell or group of cells prior to the biochemical alterations that cause the cell or group of cells within a given sample, cyst, tissue or sample to become malignant.

Any probes may be used in concert with any of the devices, systems, kits, or methods disclosed herein. As used herein, the term “probe” refers to any molecule that may bind or associate, indirectly or directly, covalently or non-covalently, to any of the substrates and/or reaction products and/or proteases disclosed herein and whose association or binding is detectable using the methods disclosed herein. In some embodiments, the probe is a fluorogenic, fluorescent, or chemiluminescent probe, an antibody, or an absorbance-based probe. In some embodiments, an absorbance-based probe, for example the chromophore pNA (para-nitroanaline), may be used as a probe for detection and/or quantification of a protease disclosed herein. In some embodiments, the probe comprises an amino acid sequence that is a substrate of an enzyme disclosed herein and/or an analog or salt thereof, including those analogs that comprise at least about 70%, at least about 75%, at least about 80%, at least about 85%, at least about 87%, at least about 90%, at least about 95%, at least about 96%, at least about 97%, at least about 98%, or at least about 99% sequence identity to amino acids SEQ ID NO: 1-9 below. A probe may be immobilized, adsorbed, or otherwise non-covalently bound to a solid surface, such that upon exposure to an enzyme for a time period sufficient to perform an enzymatic reaction, it can be enzymatically cleaved. In some embodiments, cleavage of the substrate causes a biological change in the nature or chemical availability of one or more probes such that cleavage enables detection of the reaction product. For instance, if the step of detecting comprises use of FRET, cleavage of the substrate disclosed herein causes one of the chromophore to emit a fluorescent light under exposure to a wavelength sufficient to activate such a fluorescent molecule. The intensity, length, or amplitude of a wavelength emitted from fluorescent marker can be measured and is, in some embodiments, proportional to the presence, absence or quantity of enzyme present in the reaction vessel, thereby the quantity of enzyme can be determined from detection of the intensity of or fluorescence at a known wavelength of light.

An “activity-based probe,” as used herein, refers to a certain embodiment of probe comprising a small molecule that binds to or has affinity for a molecule such as a substrate that binds an enzyme in the presence of such an enzyme, such that its bound or unbound state confers an activity readout to the enzyme. In some embodiments, the activity-based probe covalently or non-covalently binds to an enzyme or functional fragment herein. In some embodiments, the binding of the activity-based probe modifies the physical or biological activity of the enzyme. In some embodiments, the activity-based probe can be fluorescent or chemiluminescent. In some embodiments, the activity-based probe has a measurable activity of one value if the enzyme is inactive and another measurable activity if in an activated state.

As used herein, the terms “fluorogenic” and “fluorescent” probe refer to any molecule (dye, quantum dot, peptide, or fluorescent marker) that emits a known and/or detectable wavelength of light upon exposure to a known wavelength of light. In some embodiments, the substrates or peptides with known cleavage sites recognizable by any of the enzymes expressed by one or a plurality of mucinous cysts are covalently or non-covalently attached to a fluorogenic probe. In some embodiments, the attachment of the fluorogenic probe to the substrate creates a chimeric molecule capable of a fluorescent emission or emissions upon exposure of the substrate to the enzyme and the known wavelength of light, such that exposure to the enzyme creates a reaction product which is quantifiable in the presence of a fluorimeter. In some embodiments, light from the fluorogenic probe is fully quenched upon exposure to the known wavelength of light before enzymatic cleavage of the substrate and the fluorogenic probe emits a known wavelength of light, the intensity of which is quantifiable by absorbance readings or intensity levels in the presence of a fluorimeter and after enzymatic cleavage of the substrate. In some embodiments, the fluorogenic probe is a coumarin-based dye or rhodamine-based dye with fluorescent emission spectra measureable or quantifiable in the presence of or exposure to a predetermined wavelength of light. In some embodiments, the fluorogenic probe comprises rhodamine. In some embodiments, the fluorogenic probe comprises rhodamine-100. Coumarin-based fluorogenic probes are known in the art, for example in U.S. Pat. Nos. 7,625,758 and 7,863,048, which are herein incorporated by reference in their entireties. In some embodiments, the fluorogenic probes are a component to, covalently bound to, non-covalently bound to, intercalated with one or a plurality of substrates to any of the enzymes disclosed herein. In some embodiments, the fluorogenic probes are chosen from ACC or AMC. In some embodiments, the fluorogenic probe is a fluorescein molecule. In some embodiments, the fluorogenic probe is capable of emitting a resonance wave detectable and/or quantifiable by a fluorimeter after exposure to one or a plurality of enzymes disclosed herein. “Fluorescence microscopy,” which uses the fluorescence to generate an image, may be used to detect the presence, absence, or quantity of a fluorescent probe. In some embodiments, fluorescence microscopy comprises measuring fluorescence resonance energy transfer (FRET) within a FRET-based assay.

A “chemiluminescent probe” refers to any molecule (dye, peptide, or chemiluminescent marker) that emits a known and/or detectable wavelength of light as the result of a chemical reaction. Chemiluminescence differs from fluorescence or phosphorescence in that the electronic excited state is the product of a chemical reaction rather than of the absorption of a photon. Non-limiting examples of chemiluminescent probes are luciferin and aequorin molecules. In some embodiments, a chemiluminescent molecule is covalently or non-covalently attached to a substrate disclosed herein or an enzyme, such that the excited electronic state can be quantified to determine directly that an enzyme, such as an aspartyl or serine protease, is in a reaction vessel, or, indirectly, by quantifying the amount of reaction product was produced after activation of the probe on the substrate or a portion of the substrate.

As used herein, an “enzyme” can be any partially or wholly proteinaceous molecule which carries out a chemical reaction in a catalytic manner upon exposure to a substrate. Such enzymes can be native enzymes, fusion enzymes, proenzymes, apoenzymes, denatured enzymes, famesylated enzymes, ubiquitinated enzymes, fatty acylated enzymes, gerangeranylated enzymes, GPI-linked enzymes, lipid-linked enzymes, prenylated enzymes, naturally-occurring or artificially-generated mutant enzymes, enzymes with side chain or backbone modifications, enzymes having leader sequences, and enzymes complexed with non-proteinaceous material, such as proteoglycans, proteoliposomes. Enzymes can be made by any means, including natural expression, promoted expression, cloning, various solution-based and solid-based peptide syntheses, and similar methods known to those of skill in the art. Proteases of the present invention are enzymes that break down or cleave peptides.

As used herein, the term “sample” refers generally to a limited quantity of something which is intended to be similar to and represent a larger amount of that thing. In the present disclosure, a sample is a collection, fluid, blood, swab, brushing, scraping, biopsy, removed tissue, or surgical resection that is to be tested. In some embodiments, the sample is bodily fluid such as fluid from a pancreatic cyst. In some embodiments, samples are taken from a patient or subject that is believed to have a pancreatic cyst. In some embodiments, a sample believed to contain cells from a mucinous pancreatic cyst or a high grade or invasive cancer of the pancreas is compared to a “control sample” that is known not to contain mucinous cells. In some embodiments, a sample believed to comprise one or a plurality of cells derived from a mucinous pancreatic cyst is compared to a control sample that contains benign nonmucinous cells or is free of cells from a mucinous cyst. As used herein, “control sample” or “reference sample” refer to samples with a known presence, absence, or quantity of substance being measured, that is used for comparison against an experimental sample.

A “score” is a numerical value that may be assigned or generated after normalization of the value based upon the presence, absence, or quantity of substrates or enzymes disclosed herein. In some embodiments, the score is normalized in respect to a control raw data value.

The disclosure relates to a system, composition, and series of methods of using the systems and compositions for the analysis of a sample from a subject to accurately diagnose, prognose, or classify the subject with a pancreatic cyst. The disclosure also relates to a system, composition, and series of methods of using the systems and compositions for the analysis of a sample from a subject to accurately diagnose, prognose, or classify the subject with a pancreatic cyst or pancreatic cancer. The disclosure relates to a system, composition, and series of methods of using the systems and compositions for the analysis of a sample from a subject to accurately diagnose, prognose, or classify the subject with a certain highly grade or invasive pancreatic cancer or a cyst that is mucinous. In some embodiments, the system of the present invention comprises a means of detecting and/or quantifying or observing data comprising morphological features, the expression of protein, and/or the expression of nucleic acids in a plurality of cells; and correlating that data with a subject's medical history to predict clinical outcome, treatment plans, preventive medicine plans, or effective therapies. In some embodiments, the disclosure relates to methods of detecting pancreatic cancer or staging pancreatic cancer based upon the detected presence of a pancreatic ductal adenocarcinoma (PDA), an intraductal papillary mucinous neoplasm (IPMN), a mucinous cystic neoplasm (MCN), a serous cystadenoma (SCA), High Grade Dysplasia/Invasive Pancreatic Cysts or a pseudocyst. In some embodiments, the disclosure relates to methods of detecting pancreatic cancer or staging pancreatic cancer based upon the detected presence of a mucinous versus non-mucinous cyst. In some embodiments, the disclosure relates to methods of detecting pancreatic cancer or staging pancreatic cancer based upon the detected presence, absence or quantity of TPP1 either alone or in combination other biomarkers such as gastricsin and cathepsin E.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001.

Some embodiments include methods and systems with the use of Carcinoembryonic antigen (CEA). CEA is a set of highly related glycoproteins (including CD66a, CD66b, CD66c, CD66d, CD66e, and CD66f) involved in cell adhesion which are normally produced in gastrointestinal tissue during fetal development, but the production stops before birth. Therefore, CEA is usually present only at very low levels in the blood of healthy adults. However, the serum levels are raised in some types of cancer, which means that it can be used as a tumor marker in clinical tests. In some embodiments of the present invention, the presence, absence, and/or quantity of CEA of a functional isoform is detected.

The disclosure relates to methods of treating a subject in need thereof diagnosed with or suspected of having pancreatic cancer, methods of diagnosing or identifying a subject with a mucinous cyst or highgrade or invasive cyst comprising:

(a) contacting one or a plurality of substrates specific for TPP1 and/or functional fragment thereof with a sample;

(b) quantifying the amount of TPP1 and/or functional fragment thereof in the sample;

(c) calculating one or more scores based upon the presence, absence, or quantity of TPP1 and/or functional fragment thereof;

(d) correlating the one or more scores to the presence, absence, or quantity of TPP1 and/or functional fragment thereof, such that if the amount of TPP1 and/or functional fragment thereof is greater than the quantity of TPP1 and/or functional fragment thereof in a control sample, the correlating step comprises diagnosing a subject with pancreatic cancer; and

(e) administering a treatment and/or recommending or performing surgical resection of the tissue from which the sample is taken.

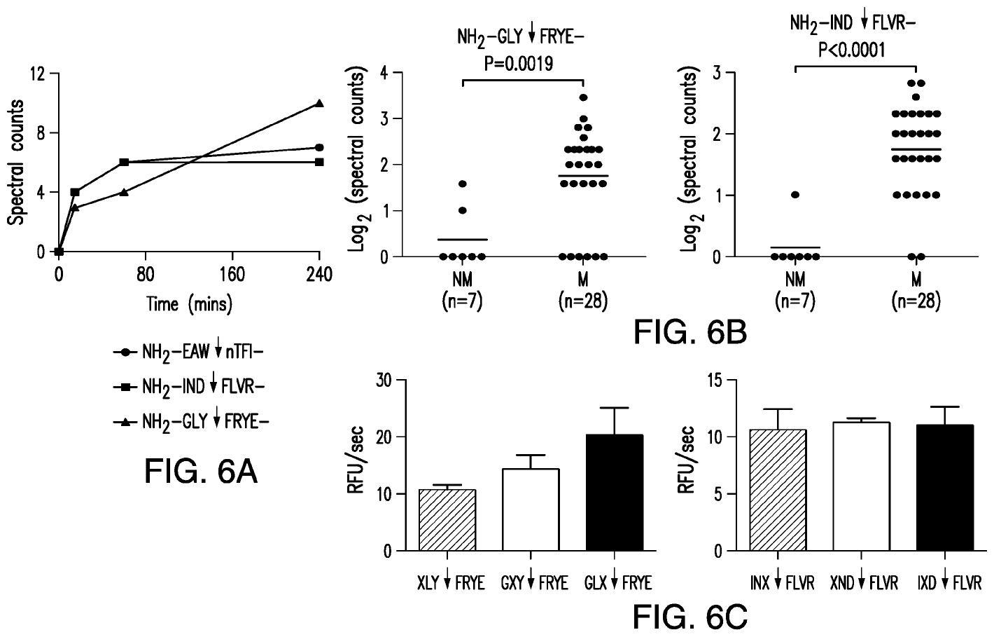

In some embodiments of any of the methods disclosed herein the substrate is one or a plurality of substrates chosen from a substrate comprising: the amino acid sequences of FIG. 6, or an analog that is at least 70%, 80%, 87%, 90%, 95%, 96%, 97%, 98%, or 99% homologous to the amino acid sequences above.

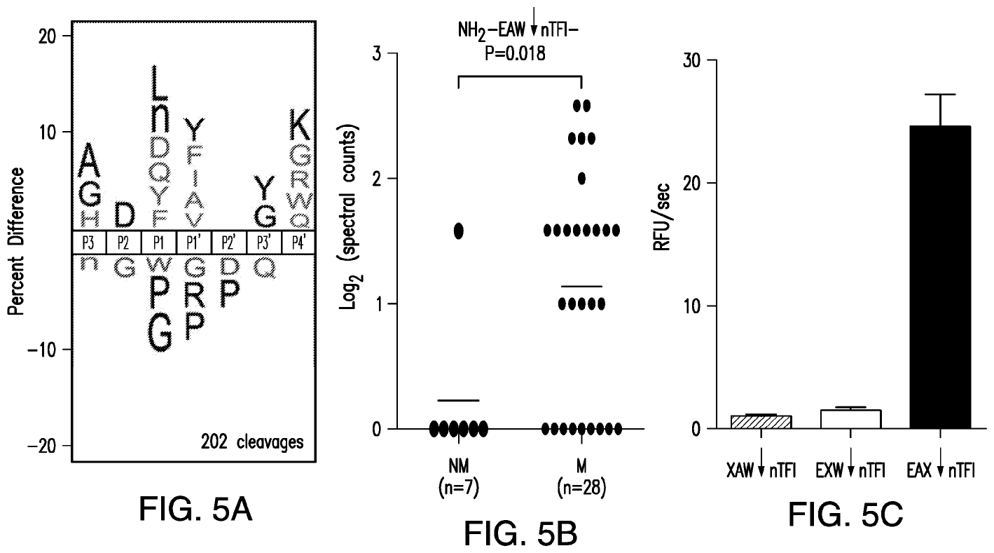

As used herein, the terms “substrate cleaved in the presence of” is a molecule comprising an amino acid sequence recognized by any enzyme disclosed herein. In some embodiments the molecule comprises an amino acid sequence recognized by TPP1 and cleaved at the amino acid sequence. In some embodiments, the molecule comprises an amino acid sequence recognized by a lysosomal serine protease disclosed herein and cleaved at that amino acid sequence. In some embodiments, the peptidyl proteases disclosed herein cut substrates at the position between the amino acids identified with a line in FIGS. 5A and 5B. In some embodiments, any of the probes comprise any one or combination of amino acid sequence identified in FIG. 5A, and, in methods in which they are used, the methods comprise a step in which diagnosing occurs by detecting the presence or quantity of reaction product formed by any of the disclosed peptidyl proteases contacting one or a combination of probes in an enzymatically effective amount sufficient to cause probe to cleave into a reaction product. In some embodiments, if the enzyme detected is an lysosomal peptidase protease, the substrate comprises X₁-X₂-X₃-X₄-X₅-X₆-X₇-X₈
wherein X₁ is D;
X₂ is E, F, W, or Y;
X₃ is G, D, M, or R;
X₄ is Y, F, W, L, or M;
X₅ is Y, M, L, A, F, or S;
X₆ is L, T or R;
X₇ is Q or Y; and
X₈ is H.

In some embodiments, the lysosomal peptidase proteases disclosed herein cut substrates between amino acids X₄ and X₅ when the following sequence features are included:
X₃-X₄-X₅-X₆
X₃ is independently selected from G, K, T, D, M, I, L S or R or a modified amino acid residue thereof;
X₄ is independently selected from Y, F, A, W, L, Y, norleucine or M or a modified amino acid residue thereof;
X₅ is independently selected from Y, M, L, A, F, T, P or S or a modified amino acid residue thereof; and
X₆ is independently selected from L, Y, G, A, T or R or a modified amino acid residue thereof

TABLE 1
Target proteases and their cleavage sites.

| Target | Sequence | Total | Relative | kcat/Km | Pubmed |
|---|---|---|---|---|---|
| Protease | (\| = site of cleavage) | Cleavage | Cleavage | (M-1/s-1) | ID |
| Gastricsin | DEGW \| ALQH | 30 | 0.47 | 4.8 x 10⁵ | |
| | VGKW \| SYRM | 22 | 0.34 | | |
| | NMKW \| TRVL | 21 | 0.33 | | |
| | PWTW \| YGVK | 64 | 1.00 | | |
| Cathepsin E | FGIF \| YLNG | 22 | 0.88 | | |
| | HMIA \| LYWG | 10 | 0.40 | | |
| | IKIL \| MFYW | 17 | 0.68 | | |
| | GLY \| FRYE | 25 | 1.00 | | |
| | AGFSL \| PA | | | 1.7 x 10⁷ | 20600629 |

In some embodiments, if the enzyme detected is a serine protease, the substrate or probe comprises the amino acids sequence identified in FIG. 5A, 5B, 5C, 6A, 6B or 6C, or combinations of thereof. In some embodiments, if the enzyme detected is a serine protease, the substrate or probe comprises the amino acids sequence X₁-X₂-X₃-X₄-X₅ as identified in the table below. In some embodiments, if the enzyme detected is a serine protease, the substrate or probe comprises the amino acids sequence X₂-X₃-X₄-X₅ as identified in the table below. In some embodiments, if the enzyme detected is a serine protease, the substrate or probe comprises the amino acids sequence X₃-X₄-X₅ as identified in the table below. In some embodiments, if the enzyme detected is a serine protease, the substrate or probe comprises the amino acids sequence X₂-X₃-X₄ as identified in the table below. In some embodiments, if the enzyme detected is a serine protease, the substrate or probe comprises the amino acids sequence X₁-X₂-X₃.

TABLE 2
Cleavage Site for Serine Proteases

| X1 | X2 | X3 | X4 | X5 |
|---|---|---|---|---|
| Arg | Nle | Leu | Phe | Val |
| His | Pro | Phe | Trp | Ile |
| Tyr | Ala | Nle | Tyr | Nle |
| Lys | Phe | Tyr | Val | Leu |
| Gln | Tyr | Trp | Ile | Trp |
| Phe | Trp | Glu | Ala | Phe |
| Ile | Ile | Asp | Thr | Tyr |
| Ala | Val | Gln | Leu | Ser |
| Ser | Thr | Ala | Nle | Thr |
| Glu | Glu | Asn | Ser | Ala |
| Thr | Leu | Arg | Glu | Arg |
| Val | Ser | Lys | Asn | Gln |
| Asn | Gly | Ser | Asp | His |
| Nle | Gln | His | His | Asn |
| Trp | Lys | Val | Arg | Lys |
| Leu | Asn | Ile | Gln | Glu |
| Gly | Asp | Thr | Gly | Asp |
| Asp | Arg | Gly | Lys | Gly |
| Pro | His | Pro | Pro | Pro |

As used herein, “specific for” or “specifically binds to” means that the binding affinity of a substrate to a specified target nucleic acid sequence, such as a tripeptidyl peptidase, is statistically higher than the binding affinity of the same substrate to a generally comparable, but non-target amino acid sequence. The substrate's Kd to each nucleotide sequence can be compared to assess the binding specificity of the substrate to a particular target nucleotide sequence.

In some embodiments, TPP1 may refer to an amino acid sequence according to SEQ ID NO: 10 below or a functional fragment thereof or variant thereof that comprises at least about 70%, 75%, 80%, 85%, 90%, 91%, 92%, 93%, 94%, 95%, 96%, 97%, 98%, or 99% sequence identity to such amino acid sequence:

(SEQ ID NO: 10)
LHLGVTPSVIRKRYNLTSQDVGSGTSNNSQACAQFLEQYFHDSDLAQFM
RLFGGNFAHQASVARVVGQQGRGRAGIEASLDVQYLMSAGANISTWVYS
SPGRHEGQEPFLQWLMLLSNESALPHVHTVSYGDDEDSLSSAYIQRVNT
ELMKAAARGLTLLFASGDSGAGCWSVSGRHQFRPTFPASSPYVTTVGGT
SFQEPFLITNEIVDYISGGGFSNVFPRPSYQEEAVTKFLSSSPHLPPSS
YFNASGRAYPDVAALSDGYWVVSNRVPIPWVSGTSASTPVFGGILSLIN
EHRILSGRPPLGFLNPRLYQQHGAGLFDVTRGCHESCLDEEVEGQGFCS
GPGWDPVTGWGTPNFPALLKTLLNP.

In some embodiments, cathepsin E may refer to an amino acid sequence according to SEQ ID NO: 11 below or a functional fragment thereof or variant thereof that comprises at least about 70%, 75%, 80%, 85%, 90%, 91%, 92%, 93%, 94%, 95%, 96%, 97%, 98%, or 99% sequence identity to such amino acid sequence:

(SEQ IDNO: 11).
MKTLLLLLLVLLELGEAQGSLHRVPLRRHPSLKKKLRARSQLSEFWKSH
NLDMIQFTESCSMDQSAKEPLINYLDMEYFGTISIGSPPQNFTVIFDTG
SSNLWVPSVYCTSPACKTHSRFQPSQSSTYSQPGQSFSIQYGTGSLSGI
IGADQVSVEGLTVVGQQFGESVTEPGQTFVDAEFDGILGLGYPSLAVGG
VTPVFDNMMAQNLVDLPMFSVYMSSNPEGGAGSELIFGGYDHSHFSGSL
NWVPVTKQAYWQIALDNIQVGGTVMFCSEGCQAIVDTGTSLITGPSDKI
KQLQNAIGAAPVDGEYAVECANLNVMPDVTFTINGVPYTLSPTAYTLLD
FVDGMQFCSSGFQGLDIHPPAGPLWILGDVFIRQFYSVFDRGNNRVGLA
PAVP

In some embodiments, gastricsin may refer to an amino acid sequence according to SEQ ID NO: 12 below or a functional fragment thereof or variant thereof that comprises at least about 70%, 75%, 80%, 85%, 90%, 91%, 92%, 93%, 94%, 95%, 96%, 97%, 98%, or 99% sequence identity to such amino acid sequence:

(SEQ ID NO: 12)
MKWMVVVLVCLQLLEAAVVKVPLKKFKSIRETMKEKGLLGEFLRTHKYD
PAWKYRFGDLSVTYEPMAYMDAAYFGEISIGTPPQNFLVLFDTGSSNLW
VPSVYCQSQACTSHSRFNPSESSTYSTNGQTFSLQYGSGSLTGFFGYDT
LTVQSIQVPNQEFGLSENEPGTNFVYAQFDGIMGLAYPALSVDEATTAM
QGMVQEGALTSPVFSVYLSNQQGSSGGAVVFGGVDSSLYTGQIYWAPVT
QELYWQIGIEEFLIGGQASGWCSEGCQAIVDTGTSLLTVPQQYMSALLQ
ATGAQEDEYGQFLVNCNSIQNLPSLTFIINGVEFPLPPSSYILSNNGYC
TVGVEPTYLSSQNGQPLWILGDVFLRSYYSVYDLGNNRVGFATAA.

Human or non-human variants of the enzymes above are contemplated by the methods, systems, and devices disclosed herein. Variants of these enzymes include sequences that are at least 70% homologous or identical to the human sequences above. As used herein, the term “variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule or amino acid sequence of the disclosure will have at least about 70%, 75%, 80%, 85%, 87%, 90%, 91%, 92%, 93%, 94%, 95%, 96%, 97%, 98%, 99% or more sequence identity to that particular polynucleotide as determined by sequence alignment programs and parameters as described elsewhere herein.

Variants of a particular nucleic acid molecule of the disclosure (i.e., the reference amino acid sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 87%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 87%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 20, 15, 10, 9, 8, 7, 6, 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly.

The disclosure relates to a system comprising a solid substrate such as a plastic plate or dish comprising at least one vessel with a sidewall and bottom surface defining a volume into which a sample may be tested. The surface comprises a substrate either covalently or noncovalently bound to the bottom surface or in suspension within the vessel, such that after exposure to an enzyme (such as a serin protease) a reaction takes place between the substrate and a TPP1 or variant thereof in a sample, a reaction product ay be detected using standard detection techniques. In some embodiments, the reaction product may be detected by an antibody.

Any probe disclosed herein may be an antibody. The term “antibody” as used herein refers to a polypeptide or group of polypeptides that are comprised of at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. As used herein, a “targeted binding agent” is an antibody, or binding fragment thereof, that preferentially binds to a target site. In one embodiment, the targeted binding agent is specific for only one target site. In other embodiments, the targeted binding agent is specific for more than one target site. In one embodiment, the targeted binding agent may be a monoclonal antibody and the target site may be an epitope. “Epitope” refers to that portion of an antigen or other macromolecule capable of forming a binding interaction that interacts with the variable region binding pocket of an antibody. “Binding fragments” of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. An antibody substantially inhibits adhesion of a receptor to a counter-receptor when an excess of antibody reduces the quantity of receptor bound to counter-receptor by at least about 20%, 40%, 60% or 80%, and more usually greater than about 85% (as measured in an in vitro competitive binding assay). An antibody may be oligoclonal, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody and antibodies that can be labeled in soluble or bound form as well as fragments, variants or derivatives thereof, either alone or in combination with other amino acid sequences provided by known techniques. An antibody may be from any species. The term antibody also includes binding fragments of the antibodies of the invention; exemplary fragments include Fv, Fab, Fab′, single stranded antibody (svFC), dimeric variable region (Diabody) and di-sulphide stabilized variable region (dsFv). As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99% sequence identity to the antibodies or immunoglobulin molecules described herein. In particular, conservative amino acid replacements are contemplated. Conservative replacements are those that take place within a family of amino acids that have related side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are an aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding function or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. See, for example, Bowie et al. Science 253:164 (1991), which is incorporated by reference in its entirety. In some embodiments, the antibody fragments, analogs thereof of the disclosure are antibodies, antibody fragments, analogs thereof that bind to a reaction fragment resulting from the cleavage of a substrate exposed to TPP1 or any other enzyme disclosed herein. In some embodiments, the antibody fragments, analogs thereof of the disclosure are antibodies, antibody fragments, analogs thereof that bind to a reaction fragment resulting from the cleavage of a substrate exposed to gastricsin, cathepsin E and/or TPP1. In some embodiments, the antibody fragments, analogs thereof of the disclosure are antibodies, antibody fragments, analogs thereof that bind to TPP1 and/or cathepsin E, and/or gastricsin. In some embodiments, the system comprises a solid substrate spotted in a patterned or non-patterned formation at discrete locations on the solid substrate by one or a plurality of substrates for TPP1 and/or gastricsin and/or cathepsin E. In other embodiments, the system comprises a solid substrate spotted in a patterned or non-patterned formation at discrete locations on the solid substrate by one or a plurality of substrates for TPP1 and/or gastricsin and/or cathepsin E; and the system further comprises one or a plurality of antibodies, antibody fragments, or analogs of the antibodies that bind to a reaction fragment resulting from the cleavage of a substrate exposed to gastricsin, cathepsin E and/or TPP1 (or a variant or functional fragment thereof) or that bind directly to gastricsin, cathepsin E and/or TPP1 (or a variant or functional fragment thereof).

In some instances, it may be desired to modify the detection probes so that they are more readily able to bind to an analyte or a reaction product. In such instances, the detection probes may be modified with certain specific binding members that are adhered thereto to form conjugated probes. For instance, the detection probe may be conjugated with antibodies as are further described below that are specific to serine and/or aspartyl proteases. The detection probe antibody may be a monoclonal or polyclonal antibody or a mixture(s) or fragment(s) thereof.

The antibodies may generally be attached to the detection probes using any of a variety of well-known techniques. For instance, covalent attachment of the antibodies to the detection probes (e.g., particles) may be accomplished using carboxylic, amino, aldehyde, bromoacetyl, iodoacetyl, thiol, epoxy and other reactive or linking functional groups, as well as residual free radicals and radical cations, through which a protein coupling reaction may be accomplished. A surface functional group may also be incorporated as a functionalized co-monomer as the surface of the detection probe may contain a relatively high surface concentration of polar groups. In addition, although detection probes are often functionalized after synthesis, such as with poly(thiophenol), the detection probes may be capable of direct covalent linking with an antibody without the need for further modification. For example, in one embodiment, the first step of conjugation is activation of carboxylic groups on the probe surface using carbodiimide. In the second step, the activated carboxylic acid groups are reacted with an amino group of an antibody to form an amide bond, The activation and/or antibody coupling may occur in a buffer, such as phosphate-buffered saline (PBS) (e.g., pH of 7.2) or 2-(N-morpholino) ethane sulfonic acid (MES) (e.g., pH of 5.3). The resulting detection probes may then be contacted with ethanolamine, for instance, to block any remaining activated sites. Overall, this process forms a conjugated detection probe, where the antibody is covalently attached to the probe. Besides covalent bonding, other attachment techniques, such as physical adsorption, may also be utilized in the present invention.

In one embodiment, the antibody may be detectably labeled by linking to an enzyme. The enzyme, in turn, when later exposed to a substrate or reaction product or enzyme disclosed herein, will react with a substrate or reaction product or enzyme disclosed herein in such a manner as to produce a chemical moiety which may be detected as, for example, by spectrophotometric or fluorometric means. Examples of enzymes which may be used to detectably label the antibodies as herein described include malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase.

In some embodiments, in vivo or in vitro methods are performed to detect the presence, absence or quantity of one or a plurality of biomarkers corresponding to the likelihood of acquiring or having a pancreatic or mucinous cyst. In some embodiments, any of the disclosed methods or series of methods comprise exposing a sample or tissue in situ with one or a plurality of antibodies, optionally tagged with a visual detection agent such as a probe or fluorophore, which has binding affinity for one or a plurality of the biomarkers disclosed herein. Antibodies suitable for practicing the methods of the invention may be monoclonal and multivalent, and may be human, humanized or chimeric antibodies, comprising single chain antibodies, Fab fragments, F(ab′) fragments, fragments produced by a Fab expression library, and/or binding fragments of any of the above. In certain embodiments of the invention, the antibodies are human antigen-binding antibody fragments of the present invention and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, CH3 and CL domains. Also included in the disclosure are antigen-binding fragments comprising any combination of variable region(s) with a hinge region, CH1, CH2, CH3 and CL domains. Preferably, the antibodies are human, murine (e.g., mouse and rat), donkey, sheep, rabbit, goal, guinea pig, camelid, horse, or chicken. As used herein, “human” antibodies include antibodies having the amino acid sequence of a human immunoglobulin and include antibodies isolated from human immunoglobulin libraries, from human B cells, or from animals transgenic for one or more human immunoglobulins.

### Systems

The present invention relates to devices and methods for measurement and staging of pancreatic pathologies. Exploiting endogenous biochemical protein expression can provide sufficient information to aid the process of high grade dysplasia or invasive carcinoma of pancreatic epithelium and to provide such as guidance for excision of mucinous cysts found in the pancreas of a subject, for example.

Embodiments of the present invention include a multi-wavelength fluorescence-based diagnostic and/or spectroscopy strategy that combines biochemical reactions elements and software components to provide a system suited for diagnosis. A further preferred embodiment utilizes Raman spectral measurements to provide diagnostic information. Multimodal illumination and detection techniques employing a combination of fluorescence, reflectance, and Raman measurements can also be used. Some embodiments can include an imaging detector, such as a CT or MRI, to image the pancreas before and/or after the biochemical detection of serine and/or aspartyl proteases in cystic fluid from a pancreatic cyst. In any of the methods disclosed herein, a procedure and one or more spectral detectors to detect fluorescence spectral data or images, Raman spectral data or images, and reflectance spectral data or images are obtained. Preferably, a Raman light source has an excitation wavelength of at least 700 nm. A spectrometer with one or more wavelength dispersing elements and one or more detector elements can be used to generate spectral data or spectral images. Additional Raman measurements including resonance Raman, surface enhanced Raman, and graphene enhanced Raman spectral measurements can be used for certain applications.

Enzymes that are aberrantly expressed in pancreatic cancers and used for biomarkers of pancreatic disease are related to the disclosure. Determining the substrate specificity of these enzymes is a critical step in unraveling their biological functions both in normal physiological processes and in disease states. Provided herein are systems for analysis of characterization of and gradation of abnormal pancreatic tissue through enzyme-substrate specificity. In some embodiments, peptide-based technologies are used for analysis of protease specificity. In some embodiments, the system comprising one or a plurality of discrete locations on a solid support upon which (i) antibodies for serine proteases or aspartyl proteases are immobilized; (ii) substrates specific to the enzymes of functional fragments thereof are immobilized; and/or (iii) a combination of the above (i) and (ii) are immobilized. In some embodiments, the serine protease is TPP-1 and the aspartyl protease is gastricsin. In some embodiments, the proteases detected in a sample are TPP-1, gastricsin, and/or cathepsin E, and substrates specific to those enzymes (or functional fragments or variants thereof) are immobilized to a solid support. In some embodiments, the discrete location is in or proximate to a well. In some embodiments, the system comprises a solid support comprising a series of wells or vessels to perform the enzymatic reaction in solution. In some embodiments, such as MSP (MS, as identified below), the system comprises substrates specific to the above-identified enzymes that are fused to or noncovalently bound to one or a plurality of probes, such that after the enzymatic reaction takes place, a probe can be used to detect the presence, absence or quantity of reaction products made as a result of the presence, absence or quantity of enzyme in the sample. In some embodiments, the reaction product are cleaved fragments of substrates, the detection of which can be used to correlate the presence, absence, detection or quantification of enzyme in the sample.

In some embodiments, the system of the disclosure comprises use of a Multiplex Substrate Profiling by Mass Spectrometry (MSP-MS) assay to determine substrate specificity. Mass spectrometry in combination with proteome-derived substrate libraries has been successfully applied to define protease specificity. These “degradomics” methods use liquid chromatography with tandem mass spectrometry (LC-MS/MS) to identify the site of proteolytic cleavage within proteome-derived peptides and can define prime side and nonprime side specificity determinants in a single assay. These techniques are quite powerful, but require chemical labeling steps for enrichment and identification of neo-termini and present a challenge in extracting kinetic parameters.

MSP-MS was developed to provide simple, yet highly sensitive and quantitative assay for assessing the extended substrate specificity of proteases. This technique currently uses a library of 228 synthetic tetradecapeptides that contain maximal physicochemical diversity within a minimal sequence space. This library was designed based on the observation that most proteases require two optimally positioned amino acids for substrate recognition and cleavage. This phenomenon is generally referred to as the “two-site hypothesis.” For example, the specificity of granzyme B is dominated by a preference for isoleucine at the P4 position and aspartic acid at the P1 position. As is evident in the crystal structure of granzyme B, two prominent cavities on the enzyme surface (S1 and S4) accommodate these residues and are the primary determinants of substrate recognition. Though these sites are not the only determinants of enzyme efficiency, they contribute to greater than 70% of the binding energy required for substrate recognition and turnover. Numerous proteases appear to follow the two site hypothesis with the sites being juxtaposed, for example, on either side of the scissile bond or separated in space along the substrate by one or two amino acids. Therefore, physicochemical diversity in the MSP-MS library was generated through incorporation of all neighbor (XY) and near-neighbor (X*Y, X**Y) amino acid pairings. This simple and chemically defined library enables facile extraction of kinetic parameters for each substrate and is readily amenable to profiling the specificity of purified proteases or complex biological samples without the need for enrichment strategies.

For MSP-MS specificity determination, a recombinant protease or other biological sample of interest is incubated with the peptide library and aliquots are removed at multiple time points. Cleavage sites within library peptides are then identified through LC-MS/MS analysis of each time point and specificity is visualized using a sequence logo, which displays protease amino acid preference relative to the site of cleavage. Label-free quantitation of both parent peptides and their corresponding cleavage products over time can be used to determine kinetic parameters for substrate hydrolysis. This information is critical for the prioritization of optimal sequences for substrate and inhibitor design.

The MSP-MS platform of the system of the disclosure comprises a direct cleavage assay that uses mass spectrometry-based peptide sequencing for detection of degradation products in a mixture of synthetic peptides. Due to the high information content relative to low background, this method requires no additional labelling or sample fractionation as do many other mass spectrometry-based techniques, and is therefore highly reproducible. Peptide degradation kinetics can be monitored by label-free quantitation of parent ion MS peaks, and biological samples containing multiple proteases was deconvoluted through the use of class-specific protease inhibitors. The MSP-MS assay relies on a physicochemically-diverse library of 14-mer peptides to directly monitor global protease activity through LC-MS/MS sequencing of peptide cleavage products. The ability of the MSP-MS method to accurately determine extended substrate specificity has been validated with over 40 proteases from all major classes. Most other substrate profiling techniques rely on peptides labeled with bulky fluorophores, which excludes analysis of exopeptidases. In contrast, the label-free nature of the peptides used in MSP-MS allows for characterization of exopeptidase activity, which has been suggested to be misregulated in malignant mucinous cysts. In addition, label-free quantitation of changes in peptide mass spectra enables the determination of enzyme kinetic parameters.

To profile cyst fluid proteolytic activity in a global, unbiased manner, the MSP-MS assay disclosed herein is used in combination with traditional proteomic and biochemical techniques. Briefly, pancreatic cyst fluid is combined with a physicochemically-diverse library of synthetic peptides as a pool of substrates for digestion. The MSP-MS assay is run at both acidic and neutral pH, to capture a broad range of activities. Based on the proteolytic signatures identified from the MSP-MS assay, an enzymatic assay system for pancreatic cyst classification is developed.

In some embodiments, the enzymatic assay system of the disclosure is a fluorescence-based enzymatic assay for pancreatic cyst classification. In some embodiments, the system of the disclosure comprises internally quenched FRET substrates that incorporated the P4 to P4′ amino acids from the specificity profile of the protease, such as TPP1. The FRET substrates contained a 7-amino-4-methylcoumarin (AMC) fluorophore and a dinitrophenol (DNP) quencher positioned at opposing termini, such that protease cleavage yields a fluorescence signal that can be easily detected with a simple benchtop microplate reader. In some embodiments, the system of the disclosure comprises an internally quenched substrate specific for TPP1 which contains the selected P3-P4′ sequences and the AMC fluorophore conjugated to the side-chain amine of a lysine residue in P1 to maintain the free N-terminal amine for recognition by TPP1. In some embodiments, the fluorophore is covalently bound to the substrates. In some embodiments, the fluorophore is non-covalently bound to the substrates. In some embodiments, the quencher is covalently bound to the substrates. In some embodiments, the quencher is non-covalently bound to the substrates.

In some embodiments, the system of the disclosure comprises one or a plurality of probes and/or stains that bind to at least one TPP1 and/or functional fragment thereof. In some embodiments, the probe and stain is one or a plurality of fluorescently labeled or chemiluminescent substrates specific for at least one TPP1 and/or functional fragment thereof. In some embodiments, the system of the disclosure comprises one or a plurality of substrates specific to at least one TPP1 and/or functional fragment thereof. In some embodiments, the one or plurality of substrates are fluorogenic, fluorescent, or chemiluminescent fluorescently labeled. In some embodiments, the one or plurality of substrates are covalently bound to a fluorescent molecule. In some embodiments, the one or plurality of substrates are non-covalently bound to a fluorescent molecule. In some embodiments, the one or plurality of substrates are internally quenched FRET substrates comprising a 7-amino-4-methylcoumarin (AMC) fluorophore and a dinitrophenol (DNP) quencher positioned at opposing termini. In some embodiments, the one or plurality of substrates are internally quenched substrates comprising the selected P3-P4′ sequences and the AMC fluorophore conjugated to the side-chain amine of a lysine residue in P1 to maintain the free N-terminal amine for recognition by TPP1. In some embodiments, the fluorophore is covalently bound to the substrates. In some embodiments, the fluorophore is non-covalently bound to the substrates. In some embodiments, the quencher is covalently bound to the substrates. In some embodiments, the quencher is non-covalently bound to the substrates.

In some embodiments, the one or plurality of substrates are chosen from a substrate having the amino acid sequence of DEGWALQH (SEQ ID NO: 1), VGKWSYRM (SEQ ID NO: 2), NMKWTRVL (SEQ ID NO: 3), PWTWYGVK (SEQ ID NO: 4), FGIFYLNG (SEQ ID NO: 5), HMIALYWG (SEQ ID NO: 6), IKILMFYW (SEQ ID NO: 7), GLYFRYE (SEQ ID NO: 8), or AGFSLPA (SEQ ID NO: 9), or a variant that comprises at least about 70%, 80%, 87%, 90%, 95%, 96%, 97%, 98%, or 99% sequence identity to the amino acid sequences above. In some embodiments, the one or plurality of substrates are chosen from a substrate having any of the amino acid sequences provided in Table 1, Table 2 and Table 5, or a variant that comprises at least about 70%, 80%, 87%, 90%, 95%, 96%, 97%, 98%, or 99% sequence identity to the amino acid sequences above. In some embodiments, the one or plurality of substrates are chosen from a substrate having any of the amino acid sequences provided in FIG. 5A, FIG. 5B, FIG. 5C, FIG. 6A, FIG. 6B and FIG. 6C, or a variant that comprises at least about 70%, 80%, 87%, 90%, 95%, 96%, 97%, 98%, or 99% sequence identity to the amino acid sequences above.

The substrates of the disclosure may be of any length including from about 7 to about 20 amino acids residues in length, from about 5 to about 30 amino acids in the length, from about 5 to about 40 amino acids in the length, from about 5 to about 30 amino acids in the length, from about 5 to about 50 amino acids in the length, from about 5 to about 60 amino acids in the length, from about 5 to about 70 amino acids in the length, from about 5 to about 80 amino acids in the length, from about 5 to about 90 amino acids in the length, from about 5 to about 100 amino acids in the length, from about 7 to about 30 amino acids in the length, from about 7 to about 15 amino acids in the length, from about 7 to about 10 amino acids in the length, or any positive integer in between those values. In some embodiments, the substrate is no more than 25, 20, 19, 18, 17, 16, 15, 14, 13, 12, 11, 10, 9, 8, 7 or six amino acids in length, but may comprise any one or plurality of probes which are detectable after the enzymes disclosed herein catalyze the production of reaction products upon contact with substrates.

In some embodiments, the one or plurality of probes, stains and/or substrates comprised in the system of the disclosure are immobilized, adsorbed, bound, or otherwise associated with a reaction vessel in a solid support or a bead, such as a magnetic bead. Solid supports can be tissue culture plates, plastic or polystyrene multiwall plates or other plastic element with one or a plurality of reaction vessels. Enzymes contained in a sample can be contacted with the one or more substrates within one or a plurality of reaction vessels on the plastic element for a time period sufficient to catalyze a reaction between the enzyme and substrate. In some embodiments, substrates may be encapsulated by or associated with nanodroplets. In some embodiments, reaction products such as cleavage product can be detected in solution or within the reaction vessel after exposure of the reaction vessel to one or a plurality of chemical stimuli for a chemiluminescent probe, visible or non-visible light that is capable of activated the electronic state of a fluorescent probe, or exposure to an antibody specific to the enzyme or substrate. In some embodiments, the substrates disclosed herein can be bound to the surface of the solid support where one or more of FRET analysis, Raman spectroscopy, mass spectroscopy, fluorescent microscopy or absorbance of light may be performed after the enzymatic reaction is complete.

Any type of solid support typically used by one of ordinary skill in the art may be used. In some embodiments, the solid support is a chip. In some embodiments, the solid support is a slide. In some embodiments, the solid support is a petri dish or polystyrene plate. In some embodiments, the solid support is a multiwell plate, including but not limited to, 12-well, 24-well, 36-well, 48-well, 96-well, 192-well, and 384-well plate.

In some embodiments, the one or plurality of probes, stains and/or substrates specific to TPP1 comprised in the system of the disclosure is in an amount that is enzymatically effective. In some embodiments, any of the systems disclosed above may further comprise one or a plurality of substrates, probes and/or stains specific for gastricsin and/or cathepsin E in an an amount that is enzymatically effective. In some embodiments, the enzymatically effective amount of substrate specific for TPP1, gastricsin and/or cathepsin E comprises from about 0.01 to about 100 μmol/L. In some embodiments, the enzymatically effective amount of substrate specific for TPP1, gastricsin and/or cathepsin E comprises from about 0.1 to about 50 μmol/L. In some embodiments, the enzymatically effective amount of substrate specific for TPP1, gastricsin and/or cathepsin E comprises from about 1 to about 40 μmol/L. In some embodiments, the enzymatically effective amount of substrate specific for TPP1, gastricsin and/or cathepsin E comprises from about 3 to about 35 μmol/L. In some embodiments, the enzymatically effective amount of substrate specific for TPP1, gastricsin and/or cathepsin E comprises from about 5 to about 30 μmol/L. In some embodiments, the enzymatically effective amount of substrate specific for TPP1, gastricsin and/or cathepsin E comprises from about 10 to about 20 μmol/L. In some embodiments, the enzymatically effective amount of substrate specific for TPP1, gastricsin and/or cathepsin E comprises about 0.1 μmol/L. In some embodiments, the enzymatically effective amount of substrate specific for TPP1, gastricsin and/or cathepsin E comprises about 0.5 μmol/L. In some embodiments, the enzymatically effective amount of substrate specific for TPP1, gastricsin and/or cathepsin E comprises about 1 μmol/L. In some embodiments, the enzymatically effective amount of substrate specific for TPP1, gastricsin and/or cathepsin E comprises about 5 μmol/L. In some embodiments, the enzymatically effective amount of substrate specific for TPP1, gastricsin and/or cathepsin E comprises about 10 μmol/L. In some embodiments, the enzymatically effective amount of substrate specific for TPP1, gastricsin and/or cathepsin E comprises about 15 μmol/L. In some embodiments, the enzymatically effective amount of substrate specific for TPP1, gastricsin and/or cathepsin E comprises about 20 μmol/L. In some embodiments, the enzymatically effective amount of substrate specific for TPP1, gastricsin and/or cathepsin E comprises about 30 μmol/L. In some embodiments, the enzymatically effective amount of substrate specific for TPP1, gastricsin and/or cathepsin E comprises about 40 μmol/L. In some embodiments, the enzymatically effective amount of substrate specific for TPP1, gastricsin and/or cathepsin E comprises about 50 μmol/L. In some embodiments, the enzymatically effective amount of substrate specific for TPP1, gastricsin and/or cathepsin E comprises about 60 μmol/L. In some embodiments, the enzymatically effective amount of substrate specific for TPP1, gastricsin and/or cathepsin E comprises about 70 μmol/L. In some embodiments, the enzymatically effective amount of substrate specific for TPP1, gastricsin and/or cathepsin E comprises about 80 μmol/L. In some embodiments, the enzymatically effective amount of substrate specific for TPP1, gastricsin and/or cathepsin E comprises about 90 μmol/L. In some embodiments, the enzymatically effective amount of substrate specific for TPP1, gastricsin and/or cathepsin E comprises about 100 μmol/L.

To facilitate the detection of a protease disclosed herein, such as a lysosomal protease, within a sample, a detectable substance may be pre-applied to a surface, for example a plate, well, bead, nanodroplet, or other solid support comprising one or a plurality of reaction vessels. In some embodiments, a sample may be pre-mixed with a diluent or reagent before it is applied to a surface. The detectable substance may function as a detection probe that is detectable either visually or by an instrumental device. Any substance generally capable of producing a signal that is detectable visually or by an instrumental device may be used as detection probes. Suitable detectable substances may include, for instance, luminescent compounds (e.g., fluorescent, phosphorescent, etc.); radioactive compounds; visual compounds (e.g., colored dye or metallic substance, such as gold); liposomes or other vesicles containing signal-producing substances; enzymes and/or substrates, and so forth. Other suitable detectable substances may be described in U.S. Pat. No. 5,670,381 to Jou, et al. and U.S. Pat. No. 5,252,459 to Tarcha, et al., which are incorporated herein in their entirety by reference thereto for all purposes. If the detectable substance is colored, the ideal electromagnetic radiation is light of a complementary wavelength. For instance, blue detection probes strongly absorb red light.

In some embodiments, the detectable substance may be a luminescent compound that produces an optically detectable signal that corresponds to the level or quantity of protease in the sample. For example, suitable fluorescent molecules may include, but are not limited to, fluorescein, europium chelates, phycobiliprotein, phycoerythrin, phycocyanin, allophycocyanin, o-phthaldehyde, fluorescamine, rhodamine, and their derivatives and analogs. Other suitable fluorescent compounds are semiconductor nanocrystals commonly referred to as “quantum dots.” For example, such nanocrystals may contain a core of the formula CdX, wherein X is Se, Te, S, and so forth. The nanocrystals may also be passivated with an overlying shell of the formula YZ, wherein Y is Cd or Zn, and Z is S or Se. Other examples of suitable semiconductor nanocrystals may also be described in U.S. Pat. No. 6,261,779 to Barbera-Guillem, et al. and U.S. Pat. No. 6,585,939 to Dapprich, which are incorporated herein in their entirety by reference thereto for all purposes.

Further, suitable phosphorescent compounds may include metal complexes of one or more metals, such as ruthenium, osmium, rhenium, iridium, rhodium, platinum, indium, palladium, molybdenum, technetium, copper, iron, chromium, tungsten, zinc, and so forth. Especially preferred are ruthenium, rhenium, osmium, platinum, and palladium. The metal complex may contain one or more ligands that facilitate the solubility of the complex in an aqueous or non-aqueous environment. For example, some suitable examples of ligands include, but are not limited to, pyridine; pyrazine; isonicotinamide; imidazole; bipyridine; terpyridine; phenanthroline; dipyridophenazine; porphyrin; porphine; and derivatives thereof. Such ligands may be, for instance, substituted with alkyl, substituted alkyl, aryl, substituted aryl, aralkyl, substituted aralkyl, carboxylate, carboxaldehyde, carboxamide, cyano, amino, hydroxy, imino, hydroxycarbonyl, aminocarbonyl, amidine, guanidinium, ureide, sulfur-containing groups, phosphorus containing groups, and the carboxylate ester of N-hydroxy-succinimide.

Porphyrins and porphine metal complexes possess pyrrole groups coupled together with methylene bridges to form cyclic structures with metal chelating inner cavities. Many of these molecules exhibit strong phosphorescence properties at room temperature in suitable solvents (e.g., water) and an oxygen-free environment. Some suitable porphyrin complexes that are capable of exhibiting phosphorescent properties include, but are not limited to, platinum (II) coproporphyrin-I and III, palladium (II) coproporphyrin, ruthenium coproporphyrin, zinc(II)-coproporphyrin-I, derivatives thereof, and so forth. Similarly, some suitable porphine complexes that are capable of exhibiting phosphorescent properties include, but not limited to, platinum(II) tetra-meso-fluorophenylporphine and palladium(II) tetra-meso-fluorophenylporphine. Still other suitable porphyrin and/or porphine complexes are described in U.S. Pat. No. 4,614,723 to Schmidt, et al.; U.S. Pat. No. 5,464,741 to Hendrix; U.S. Pat. No. 5,518,883 to Soini; U.S. Pat. No. 5,922,537 to Ewart et al.; U.S. Pat. No. 6,004,530 to Sagner, et al.; and U.S. Pat. No. 6,582,930 to Ponomarev, et al., which are incorporated herein in their entirety by reference thereto for all purposes.

Bipyridine metal complexes may also be utilized as phosphorescent compounds. Some examples of suitable bipyridine complexes include, but are not limited to, bis [(4,4′-carbomethoxy)-2,2′-bipyridine]2-[3-(4-methyl-2,2′-bipyridine-4-yl)propyl]-1,3-dioxolane ruthenium (II); bis(2,2′bipyridine)[4-(butan-1-al)-4′-methyl-2,2′-bipyridine]ruthenium (II); bis(2,2′-bipyridine)[4-(4′-methyl-2,2′-bipyridine-4′-yl)-butyric acid]ruthenium (II); tris(2,2′bipyridine)ruthenium (II); (2,2′-bipyridine) [bis-bis(1,2-diphenylphosphino)ethylene]2-[3-(4-methyl-2,2′-bipyridine-4′-yl)propyl]-1,3-dioxolane osmium (II); bis(2,2′-bipyridine)[4-(4′-methyl-2,2′-bipyridine)-butylamine]ruthenium (II); bis(2,2′-bipyridine)[1-bromo-4 (4′-methyl-2,2′-bipyridine-4-yl)butan-e]ruthenium (II); bis(2,2′-bipyridine)maleimidohexanoic acid, 4-methyl-2,2′-bipyridine-4′-butylamide ruthenium (II), and so forth. Still other suitable metal complexes that may exhibit phosphorescent properties may be described in U.S. Pat. No. 6,613,583 to Richter, et al.; U.S. Pat. No. 6,468,741 to Massey, et al.; U.S. Pat. No. 6,444,423 to Meade, et al.; U.S. Pat. No. 6,362,011 to Massey, et al.; U.S. Pat. No. 5,731,147 to Bard, et al.; and U.S. Pat. No. 5,591,581 to Massey, et al., which are incorporated herein by reference in their entireties.

In some cases, luminescent compounds may have a relatively long emission lifetime and/or may have a relatively large “Stokes shift.” The term “Stokes shift” is generally defined as the displacement of spectral lines or bands of luminescent radiation to a longer emission wavelength than the excitation lines or bands. A relatively large Stokes shift allows the excitation wavelength of a luminescent compound to remain far apart from its emission wavelengths and is desirable because a large difference between excitation and emission wavelengths makes it easier to eliminate the reflected excitation radiation from the emitted signal. Further, a large Stokes shift also minimizes interference from luminescent molecules in the sample and/or light scattering due to proteins or colloids, which are present with some body fluids (e.g., blood). In addition, a large Stokes shift also minimizes the requirement for expensive, high-precision filters to eliminate background interference. For example, in some embodiments, the luminescent compounds have a Stokes shift of greater than about 50 nanometers, in some embodiments greater than about 100 nanometers, and in some embodiments, from about 100 to about 350 nanometers.

For example, exemplary fluorescent compounds having a large Stokes shift include lanthanide chelates of samarium (Sm (III)), dysprosium (Dy (III)), europium (Eu (III)), and terbium (Tb (I)). Such chelates may exhibit strongly red-shifted, narrow-band, long-lived emission after excitation of the chelate at substantially shorter wavelengths. Typically, the chelate possesses a strong ultraviolet excitation band due to a chromophore located close to the lanthanide in the molecule. Subsequent to excitation by the chromophore, the excitation energy may be transferred from the excited chromophore to the lanthanide. This is followed by a fluorescence emission characteristic of the lanthanide. Europium chelates, for instance, have Stokes shifts of about 250 to about 350 nanometers, as compared to only about 28 nanometers for fluorescein. Also, the fluorescence of europium chelates is long-lived, with lifetimes of about 100 to about 1000 microseconds, as compared to about 1 to about 100 nanoseconds for other fluorescent labels. In addition, these chelates have narrow emission spectra, typically having bandwidths less than about 10 nanometers at about 50% emission. One suitable europium chelate is N-(p-isothiocyanatobenzyl)-diethylene triamine tetraacetic acid-Eu³.

Detectable substances (such as those capable of associating with or reacting to the presence of the reaction products cleaved by the proteases described herein), such as described above, may be used alone or in conjunction with a particle (sometimes referred to as “beads” or “microbeads”). For instance, naturally occurring particles, such as nuclei, mycoplasma, plasmids, plastids, mammalian cells (e.g., erythrocyte ghosts), unicellular microorganisms (e.g., bacteria), polysaccharides (e.g., agarose), etc., may be used. Further, synthetic particles may also be utilized. For example, in one embodiment, latex microparticles that are labeled with a fluorescent or colored dye are utilized. Although any synthetic particle may be used in the present invention, the particles are typically formed from polystyrene, butadiene styrenes, styreneacrylic-vinyl terpolymer, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchloride-acrylates, and so forth, or an aldehyde, carboxyl, amino, hydroxyl, or hydrazide derivative thereof. Other suitable particles may be described in U.S. Pat. No. 5,670,381 to Jou, et al.; U.S. Pat. No. 5,252,459 to Tarcha, et al.; and U.S. Patent Publication No. 2003/0139886 to Bodzin, et al., which are incorporated herein in their entirety by reference thereto for all purposes. Commercially available examples of suitable fluorescent particles include fluorescent carboxylated microspheres sold by Molecular Probes, Inc. under the trade names “FluoSphere” (Red 580/605) and “TransfluoSphere” (543/620), as well as “Texas Red” and 5- and 6-carboxytetramethylrhodamine, which are also sold by Molecular Probes, Inc. In addition, commercially available examples of suitable colored, latex microparticles include carboxylated latex beads sold by Bang's Laboratory, Inc. Metallic particles (e.g., gold particles) may also be utilized in the present invention.

When utilized, the shape of the particles may generally vary. In one particular embodiment, for instance, the particles are spherical in shape. However, it should be understood that other shapes are also contemplated by the present invention, such as plates, rods, discs, bars, tubes, irregular shapes, etc. In addition, the size of the particles may also vary. For instance, the average size (e.g., diameter) of the particles may range from about 0.1 nanometers to about 100 microns, in some embodiments, from about 1 nanometer to about 10 microns, and in some embodiments, from about 10 to about 100 nanometers.

In some embodiments, the system disclosed herein comprises a chip, slide or other silica surface comprising one or a plurality of addressable locations or reaction vessels within which one or a plurality of peptides, protease or peptidase substrates, and/or antibodies with an affinity for the biomarkers disclosed herein are immobilized or contained. Upon contacting a sample comprising any one of the peptides or functional fragments thereof to the one or a plurality of peptides, protease substrates, and/or antibodies with an affinity for the biomarkers disclosed herein, a reaction ensues whose reaction products are detectable by any means known in the art or disclosed herein. For instance, the reaction products may be detectable by fluorescence, optical imaging, field microscopy, mass spectrometry, or the like.

### Methods

In some embodiments, the disclosure relates to a method of detecting the presence, absence or quantity of an lysosomal peptidase and, optionally: cathepsin E and/or gastricsin; wherein the amount of lysosomal peptidase (e.g. serine or aspartyl protease) and cathepsin E and/or gastricsin in a sample is determined by calculating the amount of intensity or presence of color caused by a colorimetric substance that forms in proportion to the amount of cleaved substrate and/or serine or aspartyl protease and/or cathepsin E and/or gastricsin in a reaction vessel. Colorimteric assays may be used in vitro when a probe comprises a substrate specific for the serine or aspartyl protease and/or cathepsin E and/or gastricsin is bound, noncovalently or covalently, to a colorimetric substrate. In some embodiments, the disclosure relates to a method of detecting the presence, absence or quantity of serine or aspartyl protease and, optionally a serine protease wherein the amount of serine protease and aspartyl protease in a sample is determined by calculating the amount of intensity or presence of color caused by a colorimetric substance that forms in proportion to the amount of cleaved substrate and/or serine protease and/or aspartyl protease in a reaction vessel. Colorimteric assays may be used in vitro when a probe comprises a substrate specific for the serine protease and/or aspartyl protease is bound, noncovalently or covalently, to a colorimetric substrate.

In some embodiments, the disclosure relates to a method of detecting the presence, absence or quantity of an aspartyl or serine protease and, optionally: CEA, gastricsin and/or glucose wherein the amount of serine protease and CEA, gastricsin and/or glucose in a sample is determined by calculating the amount of intensity or presence of color caused by a colorimetric substance that forms in proportion to the amount of cleaved substrate and/or serine protease and/or CEA, gastricsin and/or glucose in a reaction vessel. Colorimteric assays may be used in vitro when a probe comprises a substrate specific for the serine protease and/or CEA, gastricsin and/or glucose is bound, noncovalently or covalently, to a colorimetric substrate. In some embodiments, the disclosure relates to a method of detecting the presence, absence or quantity of a serine protease and, optionally an aspartyl protease wherein the amount of serine protease and aspartyl protease in a sample is determined by calculating the amount of intensity or presence of color caused by a colorimetric substance that forms in proportion to the amount of cleaved substrate and/or serine protease and/or aspartyl protease in a reaction vessel. Colorimteric assays may be used in vitro when a probe comprises a substrate specific for the serine protease and/or aspartyl protease is bound, noncovalently or covalently, to a colorimetric substrate.

In some embodiments, any of the methods disclosed herein comprise a step of detecting the presence or quantity of aspartyl protease with a sensitivity of no less than about 10 nM of aspartyl protease concentration in a sample. In some embodiments, any of the methods disclosed herein comprise a step of detecting the presence or quantity of aspartyl protease with a sensitivity of no less than about 10, 11, 12, 13, 14, 15, 16, 17, 18, 19, 20, 21, 22, 23, 24, 25, 30, 35, 40, 45, 50, 55, 60, 65, 70, 75, 80, 85, 90, 91, 92, 93, 94, or 95 nM of protease concentration in a sample. In some embodiments, any of the methods disclosed herein comprise a step of detecting the presence or quantity of a serine protease with a sensitivity of no less than about 10 nM of serine protease concentration in a sample. In some embodiments, any of the methods disclosed herein comprise a step of detecting the presence or quantity of serine protease with a sensitivity of no less than about 10, 11, 12, 13, 14, 15, 16, 17, 18, 19, 20, 21, 22, 23, 24, 25, 30, 35, 40, 45, 50, 55, 60, 65, 70, 75, 80, 85, 90, 91, 92, 93, 94, or 95 nM of serine protease concentration in a sample.

In some embodiments, the disclosure relates to a method of detecting the presence, absence or quantity of lysosomal protease, including but not limited to TPP1, in a sample where the amount of enzyme in a sample is determined by calculating the amount of fluorescence of a cleaved substrate in a reaction vessel and is calculated by the expression: (F_{final}−Fᵢₙᵢₜᵢₐₗ)/Fᵢₙᵢₜᵢₐₗ, wherein F stands for relative fluorescence units (RFU) and is a standard plate reader unit, where the amount of fluorescent signal detected is linearly or substantially linearly related to the amount of enzyme in a sample. In some embodiments, a threshold amount or biologically significant amount of lysosomal protease, for the amount in a sample which may indicate a high grade of dysplasia or invasive cancerous cyst versus a benign mucinous cyst, is about a 1.19, 1.20, 1.21, 1.22, 1.23, 1.24, 1.25, 1.26. 1.27, 1.28, 1.29 fold-change in fluorescence. In some embodiments, the sensitivity of the assay is about any of the sensitivities disclosed in the Examples or Figures section of the disclosure. In some embodiments, the sensitivity can be equal to detection of enzyme at a level of about 0.125 nM within the sample. In some embodiments, the sensitivity can be equal to detection of enzyme at a level of about 0.120 nM within the sample. In some embodiments, the sensitivity can be equal to detection of enzyme at a level of about 0.110 nM within the sample. In some embodiments, the sensitivity can be equal to detection of enzyme at a level of about 0.140 nM within the sample. In some embodiments, the sensitivity can be equal to detection of enzyme at a level of about 0.130 nM within the sample. In some embodiments, the sensitivity can be equal to detection of enzyme at a level of about 0.150 nM within the sample. In some embodiments, the sensitivity can be equal to detection of enzyme at a level of about 0.225 nM within the sample. In some embodiments, the sensitivity can be equal to detection of enzyme at a level of about 0.175 nM within the sample. In some embodiments, the sensitivity can be equal to detection of enzyme at a level of about 0.125 μM within the sample. In some embodiments, the sensitivity can be equal to detection of enzyme at a level of about 0.200 μM within the sample.

In some embodiments, the method of the disclosure further comprises detecting the presence, absence or quantity of gastricsin and/or cathepsin E in the sample where the amount of gastricsin and/or cathepsin E in the sample is determined by calculating the amount of fluorescence of a cleaved substrate in the reaction vessel and is calculated by the expression: (F_{final}−Fᵢₙᵢₜᵢₐₗ)/Fᵢₙᵢₜᵢₐₗ discussed above. For both gastricsin and cathepsin E, a biologically significant amount of enzyme (e.g. for the amount in a sample which may indicate a mucinous versus non-mucinous cysts), is at least about a 1.19, 1.20, 1.21, 1.22, 1.23, 1.24, 1.25, 1.26. 1.27, 1.28, 1.29 fold-change in fluorescence. In some embodiments, the sensitivity of the assay is about any of the sensitivities disclosed in the Examples or Figures section of the disclosure. In some embodiments, the sensitivity can be equal to detection of enzyme at a level of about 0.125 μM within the sample. In some embodiments, the sensitivity can be equal to detection of enzyme at a level of about 0.120 μM within the sample. In some embodiments, the sensitivity can be equal to detection of enzyme at a level of about 0.110 μM within the sample. In some embodiments, the sensitivity can be equal to detection of enzyme at a level of about 0.140 μM within the sample. In some embodiments, the sensitivity can be equal to detection of enzyme at a level of about 0.130 μM within the sample. In some embodiments, the sensitivity can be equal to detection of enzyme at a level of about 0.150 μM within the sample. In some embodiments, the sensitivity can be equal to detection of enzyme at a level of about 0.225 μM within the sample. In some embodiments, the sensitivity can be equal to detection of enzyme at a level of about 0.175 μM within the sample. In some embodiments, the sensitivity can be equal to detection of enzyme at a level of about 0.125 μM within the sample. In some embodiments, the sensitivity can be equal to detection of enzyme at a level of about 0.200 μM within the sample. In some embodiments, the sensitivity can be equal to detection of enzyme at a level of about 0.090, 0.091, 0.92, 0.093, 0.094, 0.095 μM within the sample. In some embodiments, the sensitivity can be equal to detection of enzyme at a level of from about 10, 11, 12, 13, 14, or 15 nM to about 100 nM within the sample.

In some embodiments, the disclosure relates to methods of diagnosing and/or prognosing subjects comprising correlating the amount of at least one serine protease in a sample to the stage of disease. The present disclosure also relates to a method for characterizing the stage of development or pathology of a cyst from a subject comprising a hyperproliferative cell. In one aspect, the method of characterizing the stage of development or pathology of a cyst comprising a hyperproliferative cell comprises: (a) contacting a plurality of probes specific for TPP1 and/or functional fragment thereof with a sample; (b) quantifying the amount of TPP1 and/or functional fragment thereof in the sample; (c) calculating one or more normalized scores based upon the presence, absence, or quantity of TPP1 and/or functional fragment thereof; and (d) correlating the one or more scores to the presence, absence, or quantity of TPP1 and/or functional fragment thereof, such that if the amount of TPP1 and/or functional fragment thereof is greater than the quantity of TPP1 and/or functional fragment thereof in a control sample, the correlating step comprises characterizing the sample as comprising a cyst comprising a hyperproliferative cell.

As used herein, the term “score” refers to a single value that can be used as a component in a predictive model for the diagnosis, prognosis, and/or clinical treatment plan for a subject, wherein the single value is calculated by combining and/or normalizing raw data values with or against a control value based upon features or metrics measured in the system. In some embodiments, the score is calculated by through an interpretation function or algorithm. In some embodiments, the subject is suspected of having, is at risk of developing, or has a pancreatic cyst. In some embodiments, the score is calculated through an interpretation function or algorithm that normalizes the amount of an experimental value obtained through a test disclosed herein as compared to a control value obtained through a test disclosed herein or by a predetermined value conducted prior to conducting a test disclosed herein but corresponding to a control or normal (e.g. uninfected) value.

In some embodiments, the present disclosure also relates to a method for characterizing the stage of development or pathology of a cyst from a subject as being cancerous or malignant. In one aspect, the method of characterizing the stage of development or pathology of a cyst comprises detecting the presence, absence, or quantity of TPP1 or functional fragment thereof in a sample from the subject by contacting the sample with a probe specific for TPP1 or functional fragment thereof, and/or a substrate specific for TPP1 or functional fragment thereof. In some embodiments, the method further comprises detecting the presence, absence, or quantity of CEA or functional fragment thereof in the sample from the subject by contacting the sample with a probe specific for CEA or functional fragment thereof. In some embodiments, the method further comprises detecting the presence, absence, or quantity of cathepsin E or functional fragment thereof in the sample from the subject by contacting the sample with a probe specific for cathepsin E or functional fragment thereof, and/or a substrate specific for cathepsin E or functional fragment thereof.

In some embodiments, the disclosure relates to a method of detecting the presence of a pre-cancerous or cancerous cell in a subject comprising: (a) contacting a plurality of probes specific for TPP1 and/or CEA and/or functional fragment thereof with a sample from the subject; (b) quantifying the amount of TPP1 and/or CEA and/or functional fragment thereof in the sample; (c) calculating one or more normalized scores based upon the presence, absence, or quantity of TPP1 and/or CEA and/or functional fragment thereof; and (d) correlating the one or more scores to the presence, absence, or quantity of TPP1 and/or CEA and/or functional fragment thereof, such that if the amount of TPP1 and/or CEA and/or functional fragment thereof is greater than the quantity of cathepsin E and/or gastricsin and/or CEA and/or functional fragment thereof in a control sample, the correlating step comprises characterizing the sample as comprising a pre-cancerous or cancerous cell.

The present disclosure relates to the detecting a serine protease, such as TPP1, in a subject, the method comprising: (i) obtaining a sample from the subject; and; (ii) detecting whether serine protease, such as TPP1, is present at biologically significant levels within the sample by contacting the sample with a probe or substrate specific for serine protease, such as TPP1, and detecting binding between serine protease, such as TPP1, and the probe or substrate. In some embodiments, the biologically significant levels of serine protease, such as TPP1, and/or functional fragments thereof within a sample are at or greater than about a 1 fold change in quantity as compared to the amount of serine protease, such as TPP1, or functional fragments thereof in a control sample (for instance, a sample known to be benign or noncancerous or non-malignant). In some embodiments, the biologically significant levels of serine protease, such as TPP1, and/or functional fragments thereof within a sample are at or greater than about a 1.1 fold change as compared to the amount of serine protease, such as TPP1, or functional fragments thereof in a control sample (for instance, a sample known to be benign or noncancerous or non-malignant). In some embodiments, the biologically significant levels of serine protease, such as TPP1, and/or functional fragments thereof within a sample are at or greater than about a 1.2 fold change as compared to the amount of serine protease, such as TPP1, or functional fragments thereof in a control sample (for instance, a sample known to be benign or noncancerous or non-malignant). In some embodiments, the biologically significant levels of serine protease, such as TPP1, and/or functional fragments thereof within a sample are at or greater than about a 1.3 fold change as compared to the amount of serine protease, such as TPP1, or functional fragments thereof in a control sample (for instance, a sample known to be benign or noncancerous or non-malignant). In some embodiments, the biologically significant levels of serine protease, such as TPP1, and/or functional fragments thereof within a sample are at or greater than about a 1.4 fold change as compared to the amount of serine protease, such as TPP1, or functional fragments thereof in a control sample (for instance, a sample known to be benign or noncancerous or non-malignant). In some embodiments, the biologically significant levels of serine protease, such as TPP1, and/or functional fragments thereof within a sample are at or greater than about a 1.5 fold change as compared to the amount of serine protease, such as TPP1, or functional fragments thereof in a control sample (for instance, a sample known to be benign or noncancerous or non-malignant).

In some embodiments, any tissue or body fluid sample may be used to detect the absence or presence of a mucinous cyst. Cystic fluid, saliva, cheek swabs (buccal swabs), hair bulb, blood serum and whole blood samples are among the common forms of samples used to obtain such samples. Examples of other samples can include semen, vaginal fluid, urine, lymph fluid, cerebral spinal fluid, amniotic fluid, skin and surgically excised tissue. In some embodiments, the sample is cystic fluid. One skilled in the art would readily recognize other types of samples of methods of obtaining them. In some embodiments of the methods disclosed herein, any of the methods disclosed herein comprise a step of obtaining a sample from a subject such as a human patient.

Various formats may be used to test for the presence, absence or quantity of a lysosomal protease or functional fragment thereof using the assay devices or system of the present disclosure. For instance, a “sandwich” format typically involves mixing the test sample with probes conjugated with a specific binding member (e.g., antibody) for the analyte to form complexes between the analyte and the conjugated probes. These complexes are then allowed to contact a receptive material (e.g., antibodies) immobilized within the detection zone. Binding occurs between the analyte/probe conjugate complexes and the immobilized receptive material, thereby localizing “sandwich” complexes that are detectable to indicate the presence of the analyte. This technique may be used to obtain quantitative or semi-quantitative results. Some examples of such sandwich-type assays are described by U.S. Pat. No. 4,168,146 to Grubb, et al. and U.S. Pat. No. 4,366,241 to Tom, et al., which are incorporated herein in their entirety by reference thereto for all purposes. In a competitive assay, the labeled probe is generally conjugated with a molecule that is identical to, or an analog of, the analyte. Thus, the labeled probe competes with the analyte of interest for the available receptive material. Competitive assays are typically used for detection of analytes such as haptens, each hapten being monovalent and capable of binding only one antibody molecule. Examples of competitive immunoassay devices are described in U.S. Pat. No. 4,235,601 to Deutsch, et al., U.S. Pat. No. 4,442,204 to Liotta, and U.S. Pat. No. 5,208,535 to Buechler, et al., which are incorporated herein in their entirety by reference thereto for all purposes. Various other device configurations and/or assay formats are also described in U.S. Pat. No. 5,395,754 to Lambotte, et al.; U.S. Pat. No. 5,670,381 to Jou, et al.; and U.S. Pat. No. 6,194,220 to Malick, et al., which are incorporated herein in their entirety by reference thereto for all purposes.

Although various assay device configuration have been described herein, it should be understood that any known assay device may be utilized that is capable of incorporating an antibody in accordance with the present invention. For example, electrochemical affinity assay devices may also be utilized, which detect an electrochemical reaction between a lysosomal protease (or complex thereof) and a capture ligand on an electrode strip. For example, various electrochemical assays and assay devices are described in U.S. Pat. No. 5,508,171 to Walling, et al.; U.S. Pat. No. 5,534,132 to Vreeke, et al.; U.S. Pat. No. 6,241,863 to Monbouquette; U.S. Pat. No. 6,270,637 to Crismore, et al.; U.S. Pat. No. 6,281,006 to Heller, et al.; and U.S. Pat. No. 6,461,496 to Feldman, et al., which are incorporated herein in their entirety by reference thereto for all purposes.

One skilled in the art will readily appreciate the wide range of methods and techniques used for detecting the presence and/or quantity of proteins, enzymes and/or cleavage products in a complex sample. Techniques for detecting proteins or cleavage products include, but are not limited to, microscopy, immunostaining, immunoprecipitation, immunoelectrophoresis, Western blot, BCA assays, spectrophotometry, enzymatic assays, microchip assays, and mass spectrometry. In some embodiments, purification of proteins are necessary before detection of quantification techniques are employed. Techniques for purifying proteins include, but are not limited to, chromatography methods, including ion exchange, size-exclusion, and affinity chromatography, gel electrophoresis, magnetic beads comprising any antibody, antibody-like protein or antibody fragment or variant, Bradford protein assays. In some embodiments, methods of measuring the presence, absence, or quantity of aspartyl protease or functional fragments thereof comprise antibodies or antibody fragments specific to aspartyl protease or functional fragments thereof.

In some embodiments, the disclosure relates to a method of treating a subject in need thereof diagnosed with or suspected of having pancreatic cancer or pancreatic cyst, comprising: (a) contacting one or a plurality of substrates specific for TPP1 and/or functional fragment thereof with a sample; (b) quantifying the amount of TPP1 and/or functional fragment thereof in the sample; (c) calculating one or more scores based upon the presence, absence, or quantity of TPP1 and/or functional fragment thereof (d) correlating the one or more scores to the presence, absence, or quantity of TPP1 and/or functional fragment thereof, such that if the amount of TPP1 and/or functional fragment thereof is greater than the quantity of TPP1 and/or functional fragment thereof in a control sample, the correlating step comprises diagnosing a subject with pancreatic cancer; and (e) administering to the subject a therapeutically effective amount of treatment for the pancreatic cancer. In some embodiments, the substrate is one or a plurality of substrates chosen from a substrate with formula: DEGWALQH (SEQ ID NO: 1), VGKWSYRM (SEQ ID NO: 2), NMKWTRVL (SEQ ID NO: 3), PWTWYGVK (SEQ ID NO: 4), FGIFYLNG (SEQ ID NO: 5), HMIALYWG (SEQ ID NO: 6), IKILMFYW (SEQ ID NO: 7), GLYFRYE (SEQ ID NO: 8), or AGFSLPA (SEQ ID NO: 9), any substrate identified in Table 1, Table 2, Table 5, FIG. 5A, FIG. 5B, FIG. 5C, FIG. 6A, FIG. 6B and FIG. 6C, or an analog that comprising at least about 70%, 80%, 87%, 90%, 95%, 96%, 97%, 98%, or 99% sequence homology to the amino acid sequences above.

Depending on the type and stage of the cancer and other factors, treatment options for people with pancreatic cancer can include: surgery, ablation or embolization treatments, radiation therapy, chemotherapy, targeted therapy, and immunotherapy. In some embodiments, the step of administering is surgically resecting or surgically removing the pancreatic cyst. In some embodiments, the step of administering comprises radiation therapy. In some embodiments, the step of administering comprises chemotherapy. In some embodiments, the step of administering comprises targeted therapy. In some embodiments, the step of administering comprises immunotherapy.

In some embodiments, the disclosure relates to a method of diagnosing a subject with a mucinous cyst comprising: (a) detecting a presence or quantifying an amount of TPP1 and/or functional fragment thereof, in a sample of the subject, by contacting the sample with a substrate specific for TPP1 and/or functional fragment thereof; and (b) diagnosing a subject with a mucinous cyst when the presence or quantity of TPP1 and/or functional fragment thereof is detected or quantified. In some embodiments, the step of detecting a presence or quantifying an amount of TPP1 and/or functional fragment thereof is preceded by a step of obtaining the sample from the subject. In some embodiments, step (a) of the disclosed method further comprises calculating one or more scores based upon the presence, absence, or quantity of TPP1 and/or functional fragment thereof; and step (b) of the disclosed method further comprises correlating the one or more scores to the presence, absence, or quantity of TPP1 and/or functional fragment thereof, such that, if the amount of TPP1 and/or functional fragment thereof is greater than the quantity of TPP1 and/or functional fragment thereof in a control sample; or, if the amount of TPP1 and/or functional fragment thereof is substantially equal to the quantity of TPP1 and/or functional fragment thereof in a sample taken from a subject known to have a mucinous cyst, then the subject is diagnosed as having a mucinous cyst.

In some embodiments, the subject is diagnosed as having a mucinous cyst when the amount of TPP1 and/or functional fragment thereof is at least about 1.2-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 1.3-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 1.4-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 1.5-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 1.6-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 1.7-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 1.8-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 1.9-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 2-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 2.1-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 2.2-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 2.3-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 2.4-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 2.5-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 2.6-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 2.7-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 2.8-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 2.9-fold greater than the quantity of TPP1 and/or functional fragment thereof in a control sample (e.g., from a healthy subject). In some embodiments, the subject is diagnosed as having a mucinous cyst when the amount of TPP1 and/or functional fragment thereof is at least about 3.0-fold greater than the quantity of TPP1 and/or functional fragment thereof in a control sample (e.g., from a healthy subject).

In some embodiments, the subject is diagnosed as having a mucinous cyst when the amount of TPP1 and/or functional fragment thereof detected in the sample is from about 200 pg/mL to about 500 pg/mL. In some embodiments, the subject is diagnosed as having a mucinous cyst when the amount of TPP1 and/or functional fragment thereof detected in the sample is from about 220 pg/mL to about 450 pg/mL. In some embodiments, the subject is diagnosed as having a mucinous cyst when the amount of TPP1 and/or functional fragment thereof detected in the sample is from about 250 pg/mL to about 400 pg/mL. In some embodiments, the subject is diagnosed as having a mucinous cyst when the amount of TPP1 and/or functional fragment thereof detected in the sample is from about 275 pg/mL to about 350 pg/mL. In some embodiments, the subject is diagnosed as having a mucinous cyst when the amount of TPP1 and/or functional fragment thereof detected in the sample is from about 300 pg/mL to about 350 pg/mL.

In some embodiments, the subject is diagnosed as having a mucinous cyst when the amount of TPP1 and/or functional fragment thereof detected in the sample is at least about 200 pg/mL. In some embodiments, the subject is diagnosed as having a mucinous cyst when the amount of TPP1 and/or functional fragment thereof detected in the sample is at least about 220 pg/mL. In some embodiments, the subject is diagnosed as having a mucinous cyst when the amount of TPP1 and/or functional fragment thereof detected in the sample is at least about 250 pg/mL. In some embodiments, the subject is diagnosed as having a mucinous cyst when the amount of TPP1 and/or functional fragment thereof detected in the sample is at least about 275 pg/mL. In some embodiments, the subject is diagnosed as having a mucinous cyst when the amount of TPP1 and/or functional fragment thereof detected in the sample is at least about 300 pg/mL. In some embodiments, the subject is diagnosed as having a mucinous cyst when the amount of TPP1 and/or functional fragment thereof detected in the sample is at least about 325 pg/mL. In some embodiments, the subject is diagnosed as having a mucinous cyst when the amount of TPP1 and/or functional fragment thereof detected in the sample is at least about 350 pg/mL. In some embodiments, the subject is diagnosed as having a mucinous cyst when the amount of TPP1 and/or functional fragment thereof detected in the sample is at least about 450 pg/mL. In some embodiments, the subject is diagnosed as having a mucinous cyst when the amount of TPP1 and/or functional fragment thereof detected in the sample is at least about 500 pg/mL.

In some embodiments, the disclosed method further comprises (c) detecting a presence or quantifying an amount of cathepsin E and/or gastricsin and/or functional fragment thereof, in the sample of the subject, by contacting the sample with a substrate specific for cathepsin E and/or gastricsin and/or functional fragment thereof; and (d) diagnosing a subject with a mucinous cyst when the presence or quantity of cathepsin E and/or gastricsin and/or functional fragment thereof is detected or quantified.

In some embodiments, the subject is diagnosed as having a mucinous cyst when the amount of cathepsin E and/or functional fragment thereof is at least about 1.2-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 1.3-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 1.4-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 1.5-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 1.6-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 1.7-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 1.8-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 1.9-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 2-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 2.1-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 2.2-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 2.3-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 2.4-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 2.5-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 2.6-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 2.7-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 2.8-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 2.9-fold greater than the quantity of cathepsin E and/or functional fragment thereof in a control sample (e.g., from a healthy subject). In some embodiments, the subject is diagnosed as having a mucinous cyst when the amount of cathepsin E and/or functional fragment thereof is at least about 3.0-fold greater than the quantity of cathepsin E and/or functional fragment thereof in a control sample (e.g., from a healthy subject).

In some embodiments, the subject is diagnosed as having a mucinous cyst when the amount of gastricsin and/or functional fragment thereof is at least about 2-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 2.5-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 3-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 3.5-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 4-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 4.5-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 5-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 5.5-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 6-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 6.5-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 7-fold greater than the quantity of gastricsin and/or functional fragment thereof in a control sample (e.g., from a healthy subject).

In some embodiments, the subject is diagnosed as having a mucinous cyst when the amount of gastricsin and/or functional fragment thereof is at least about 1.2-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 1.5-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 1.6-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 1.7-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 1.8-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 1.9-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 2-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 2.5-fold greater than the quantity of gastricsin and/or functional fragment thereof in a control sample (e.g., from a healthy subject). In some embodiments, the subject is diagnosed as having a mucinous cyst when the amount of gastricsin and/or functional fragment thereof is at least about 3-fold greater than the quantity of gastricsin and/or functional fragment thereof in a control sample (e.g., from a healthy subject).

In some embodiments, the subject is diagnosed as having a mucinous cyst when the amount of cathepsin E and/or gastricsin and/or functional fragment thereof detected in the sample is from about 200 pg/mL to about 500 pg/mL. In some embodiments, the subject is diagnosed as having a mucinous cyst when the amount of cathepsin E and/or gastricsin and/or functional fragment thereof detected in the sample is from about 220 pg/mL to about 450 pg/mL. In some embodiments, the subject is diagnosed as having a mucinous cyst when the amount of cathepsin E and/or gastricsin and/or functional fragment thereof detected in the sample is from about 250 pg/mL to about 400 pg/mL. In some embodiments, the subject is diagnosed as having a mucinous cyst when the amount of cathepsin E and/or gastricsin and/or functional fragment thereof detected in the sample is from about 275 pg/mL to about 350 pg/mL. In some embodiments, the subject is diagnosed as having a mucinous cyst when the amount of cathepsin E and/or gastricsin and/or functional fragment thereof detected in the sample is from about 300 pg/mL to about 350 pg/mL.

In some embodiments, the subject is diagnosed as having a mucinous cyst when the amount of cathepsin E and/or gastricsin and/or functional fragment thereof detected in the sample is at least about 200 pg/mL. In some embodiments, the subject is diagnosed as having a mucinous cyst when the amount of cathepsin E and/or gastricsin and/or functional fragment thereof detected in the sample is at least about 220 pg/mL. In some embodiments, the subject is diagnosed as having a mucinous cyst when the amount of cathepsin E and/or gastricsin and/or functional fragment thereof detected in the sample is at least about 250 pg/mL. In some embodiments, the subject is diagnosed as having a mucinous cyst when the amount of cathepsin E and/or gastricsin and/or functional fragment thereof detected in the sample is at least about 275 pg/mL. In some embodiments, the subject is diagnosed as having a mucinous cyst when the amount of cathepsin E and/or gastricsin and/or functional fragment thereof detected in the sample is at least about 300 pg/mL. In some embodiments, the subject is diagnosed as having a mucinous cyst when the amount of cathepsin E and/or gastricsin and/or functional fragment thereof detected in the sample is at least about 325 pg/mL. In some embodiments, the subject is diagnosed as having a mucinous cyst when the amount of cathepsin E and/or gastricsin and/or functional fragment thereof detected in the sample is at least about 350 pg/mL. In some embodiments, the subject is diagnosed as having a mucinous cyst when the amount of cathepsin E and/or gastricsin and/or functional fragment thereof detected in the sample is at least about 450 pg/mL. In some embodiments, the subject is diagnosed as having a mucinous cyst when the amount of cathepsin E and/or gastricsin and/or functional fragment thereof detected in the sample is at least about 500 pg/mL.

Methods of the disclosure relate to a method of identifying whether a pancreatic cyst as mucinous versus non-mucinous and identifying whether the mucinous cyst is malignant or characterized with a high degree of dysplasia. Methods also include method of treatment of a subject for pancreatic cancer, the methods comprising: (a) detecting the quantity of gastricsin and/or cathepsin E, or a functional fragment or variant thereof, in a sample of cystic fluid; (b) correlating the quantity of gastricsin, cathepsin E or functional fragment or variant thereof to whether the cyst from which the sample was obtained is mucinous; (c) detecting the quantity of TPP-1, functional fragments or variant thereof, in the sample; and (d) correlating the quantity of TPP-1, or functional fragments or variants thereof to whether the mucinous cyst of pre-cancerous, cancerous (e.g. whether characterized as HGD or IC); and (e), if cyst is characterized as cancerous of comprising HGD, administering treatment for pancreatic cancer. In some embodiments, the treatment is recommended for surgical removal of the cyst from which the cystic fluid or the treatment is surgical removal of the cyst from which the cystic fluid was obtained,

Any and all journal articles, patent applications, issued patents, or other cited references disclosed herein (including GenBank Accession numbers or other genetic information identification tags dated as of the date of the application filing) are incorporated by reference in their respective entireties. Any of the systems, methods or devices disclosed herein comprising proteases may be made or performed or used with any of the proteases disclosed herein. In some embodiments, the systems, methods or devices disclosed herein are selectively free of any one or combination of enzymes disclosed herein.

### EXAMPLES

### Example 1: The Lysosomal Aminopeptidase Tripeptidyl Peptidase 1 Displays Increased Activity in Malignant Pancreatic Cysts

Incidental detection of pancreatic cysts has increased dramatically over the last decade, but risk stratification and clinical management remain a challenge. Mucinous cysts are precursor lesions to pancreatic cancer, however, the majority are indolent. Current diagnostics cannot identify mucinous cysts that harbor cancer or reliably differentiate these lesions from nonmucinous cysts, which present minimal risk of malignant progression. We previously determined that activity of two aspartyl proteases, gastricsin and cathepsin E, was highly increased in mucinous cysts. Using a global protease activity profiling technology, termed multiplex substrate profiling by mass spectrometry (MSP-MS), we now show that aminopeptidase activity is also elevated in mucinous cysts. Proteomic analysis identified the lysosomal serine protease, tripeptidyl peptidase 1 (TPP1), in cyst fluid and parallel reaction monitoring-based mass spectrometry demonstrated that this protease was significantly more abundant in mucinous cysts. In a cohort of 110 cyst fluid samples, TPP1 activity was increased more than 3-fold in mucinous cysts relative to nonmucinous cysts. Moreover, TPP1 activity is primarily associated with mucinous cysts that harbor high-grade dysplasia or invasive carcinoma. Measurement of TPP1 activity may improve early detection and treatment of these high-risk pancreatic cysts.

1. Materials and Methods

i. Sample Acquisition

The present study included 110 cyst fluid samples from patients seen at the University of California San Francisco (San Francisco, Calif.), the University of Pittsburgh Medical Center (Pittsburgh, Pa.), Indiana University School of Medicine (Indianapolis, Ind.), and Stanford University School of Medicine (Stanford, Calif.). All patients were preconsented under institutional review board approved protocols. Only samples from patients that underwent surgical resection and pathological examination of their cystic lesion were included. Patient information is summarized in Table 3. Information includes the cyst type, highest degree of dysplasia, method of collection, and institution. All samples were stored at −80° C. prior to analysis and subject to a maximum of two freeze-thaw cycles.

TABLE 3
Characteristics of patients analyzed in this study. Patient
information including cyst type, highest grade of dyplasia
observed during histological evaluation, cyst size, collection
method, patient age, and gender.

| | | | Collection | | |
|---|---|---|---|---|---|
| | Highest | Cyst | method | | |
| | grade of | size | (EUS/ | | |
| Diagnosis | dysplasia | (mm) | Surgery) | Age | Gender |
| IPMN | High | 22 | Surgery | 60 | Female |
| IPMN | High | 19 | Surgery | 69 | Male |
| IPMN | Low | 8 | Surgery | 55 | Female |
| IPMN | High | 59 | Surgery | 78 | Male |
| IPMN | High | 26 | Surgery | 69 | Female |
| IPMN | Low | 65 | EUS | 68 | Male |
| IPMN | High | 39 | Surgery | 72 | Female |
| IPMN | High | 120 | Surgery | 78 | Male |
| IPMN | High | 18 | Surgery | 64 | Male |
| IPMN | High | 30 | Surgery | 79 | Female |
| IPMN | High | 25 | Surgery | 68 | Male |
| IPMN | Low | 18 | Surgery | 41 | Male |
| IPMN | Low | 31 | Surgery | 47 | Female |
| IPMN | High | 20 | Surgery | 51 | Male |
| IPMN | Low | 47 | Surgery | 52 | Female |
| IPMN | Low | 25 | Surgery | 53 | Female |
| IPMN | Low | 38 | Surgery | 54 | Female |
| IPMN | Low | 40 | Surgery | 62 | Female |
| IPMN | Low | 47 | Surgery | 64 | Male |
| IPMN | Low | 30 | Surgery | 69 | Female |
| IPMN | Low | 65 | Surgery | 77 | Female |
| IPMN | High | 27 | Surgery | 85 | Female |
| IPMN | High | NA | EUS | 69 | Female |
| IPMN | High | 35 | ERCP | 75 | Male |
| IPMN | High | 57 | Surgery | 67 | Female |
| IPMN | High | 120 | Surgery | 78 | Male |
| IPMN | High | NA | Surgery | 66 | Female |
| IPMN | High | 50 | Surgery | 74 | Male |
| IPMN | High | 23 | Surgery | 58 | Male |
| IPMN | High | NA | Surgery | 71 | Female |
| IPMN | Low | 23 | Surgery | 68 | Female |
| IPMN | Low | 25 | Surgery | 79 | Male |
| IPMN | High | 11 | Surgery | 72 | Male |
| IPMN | High | 30 | Surgery | 63 | Female |
| IPMN | Low | 37 | Surgery | 80 | Female |
| IPMN | Low | 20 | Surgery | 40 | Female |
| IPMN | High | 20 | Surgery | 62 | Male |
| IPMN | Low | 35 | Surgery | 84 | Male |
| IPMN | Low | 4 | Surgery | 71 | Female |
| IPMN | Low | 45 | EUS | 78 | Male |
| IPMN | Low | 60 | Surgery | 60 | Male |
| IPMN | Low | 35 | Surgery | 84 | Female |
| IPMN | Low | 30 | Surgery | 74 | Female |
| IPMN | Low | 90 | Surgery | 72 | Male |
| IPMN | Low | 50 | Surgery | 71 | Female |
| MCN | Low | 23 | Surgery | 57 | Female |
| MCN | High | 22 | Surgery | 74 | Female |
| MCN | High | 57 | Surgery | 54 | Female |
| MCN | High | 125 | Surgery | 47 | Female |
| MCN | Low | 60 | Surgery | 48 | Male |
| MCN | Low | 100 | Surgery | 44 | Female |
| MCN | Low | 140 | Surgery | 22 | Female |
| MCN | Low | 125 | Surgery | 44 | Female |
| MCN | Low | 30 | Surgery | 65 | Female |
| MCN | Low | 45 | Surgery | 49 | Female |
| MCN | High | 152 | Surgery | 42 | Female |
| MCN | Low | 65 | Surgery | 25 | Female |
| MCN | Low | 30 | Surgery | 76 | Female |
| MCN | Low | 70 | Surgery | 31 | Female |
| MCN | Low | 100 | Surgery | 42 | Female |
| MCN | High | 215 | Surgery | 50 | Female |
| MCN | Low | 19 | Surgery | 59 | Female |
| MCN | Low | 115 | Surgery | 74 | Female |
| MCN | Low | 30 | Surgery | 75 | Female |
| MCN | Low | 18 | Surgery | 61 | Female |
| MCN | Low | 50 | Surgery | 25 | Female |
| MCN | Low | 25 | Surgery | 24 | Female |
| MCN | Low | 40 | Surgery | 24 | Female |
| MCN | High | 28 | Surgery | 44 | Male |
| MCN | Low | 30 | Surgery | 50 | Female |
| MCN | Low | 50 | Surgery | 57 | Female |
| Pseudocyst | NA | 55 | EUS | 48 | Male |
| Pseudocyst | NA | 219 | Surgery | 30 | Female |
| Pseudocyst | NA | 40 | EUS | 62 | Female |
| Pseudocyst | NA | 90 | EUS | 57 | Male |
| Pseudocyst | NA | 12 | Surgery | 55 | Female |
| Pseudocyst | NA | 100 | Surgery | 56 | Female |
| Pseudocyst | NA | 90 | Surgery | 60 | Male |
| Pseudocyst | NA | 79 | EUS | 43 | Male |
| Pseudocyst | NA | 98 | EUS | 49 | Male |
| Pseudocyst | NA | 91 | EUS | 63 | Male |
| Pseudocyst | NA | 100 | Surgery | 70 | Male |
| Pseudocyst | NA | 31 | EUS | 78 | Female |
| Pseudocyst | NA | NA | EUS | 68 | Female |
| Pseudocyst | NA | NA | Surgery | 59 | Female |
| Pseudocyst | NA | NA | EUS | 67 | Male |
| Pseudocyst | NA | NA | EUS | 60 | Male |
| Pseudocyst | NA | NA | Surgery | 52 | Male |
| Pseudocyst | NA | NA | Surgery | 66 | Female |
| Pseudocyst | NA | NA | Surgery | 42 | Female |
| Pseudocyst | NA | NA | Surgery | 24 | Female |
| Pseudocyst | NA | NA | Surgery | 58 | Female |
| SCA | NA | 20 | Surgery | 58 | Male |
| SCA | NA | 160 | Surgery | 62 | Male |
| SCA | NA | 50 | EUS | 59 | Male |
| SCA | NA | 38 | EUS | 45 | Female |
| SCA | NA | 65 | Surgery | 70 | Female |
| SCA | NA | 100 | Surgery | 43 | Female |
| SCA | NA | 60 | Surgery | 66 | Male |
| SCA | NA | 105 | Surgery | 47 | Male |
| SCA | NA | 60 | Surgery | 75 | Female |
| SCA | NA | 40 | Surgery | 55 | Male |
| SCA | NA | 110 | EUS | 33 | Female |
| SCA | NA | 25 | EUS | 55 | Female |
| SCA | NA | 45 | Surgery | 26 | Female |
| SCA | NA | 25 | Surgery | 30 | Female |
| SCA | NA | 12 | Surgery | 41 | Female |
| SCA | NA | 82 | Surgery | 76 | Female |
| SCA | NA | 93 | Surgery | 76 | Male |
| SCA | NA | 30 | Surgery | 74 | Female |

ii. Multiplex Substrate Profiling by Mass Spectrometry Assay

The MSP-MS assay was performed as described previously (Ivry et al., 2017; O'Donoghue et al., 2012). Cyst fluid protein concentration was first determined through the BCA assay (Thermo Fisher, 23225). Cyst fluid samples were then diluted to 100 μg/mL in pH 3.5 acetate buffer or pH 7.5 phosphate buffer. The 228 tetradecapeptide was split into two pools of 114 peptides each and diluted to 1 μmol/L in either acetate or phosphate buffer. Equal volumes of diluted cyst fluid and peptide pools were combined and incubated at room temperature. After 15 and 60 minutes, 30 μL aliquots were removed and protease activity was quenched with 8 mol/L guanidinium hydrochloride. Aliquots were then desalted using C18 tips (Rainin). The following inhibitors were included in specified MSP-MS assays: 1 mmol/L AEBSF (Sigma, A8456), 2 mmol/L E64 (Sigma, E3132), 2 μmol/L pepstatin (Sigma, P5318), 2 mmol/L 1,10-phenanthroline (Sigma, 131337), and 10 μmol/L AAF-CMK (Enzo, BML-PI123).

Mass spectrometry analysis was performed with an LTQ Orbitrap XL Mass Spectrometer (Thermo Fisher) coupled to an Ultra Performance Liquid Chromatography (UPLC) System (Waters). Peptides were separated over a C18 column (Thermo Fisher, ES800) with a 65-minute linear gradient from 2%-30% acetonitrile. MS spectra were acquired over an m/z range of 325-1,500 and MS/MS spectra were obtained for the six most intense precursor ions by collision-induced dissociation (CID). Peak lists were generated using MSConvert and searched in Protein Prospector v. 5.10.0 against a database containing the sequences from the 228 tetradecapeptide library. Searches used a mass tolerance of 20 ppm for precursors and 0.8 Da for fragments. The following variable modifications were allowed: N-terminal pyroglutamate conversion from glutamine or glutamate and oxidation of tryptophan, proline, and tyrosine. Search outputs were then processed using the MSP-xtractor software (http://www.craiklab.ucsfedu/extractor.html). This software extracts the P4-P4′ sequences and spectral counts for all identified cleavages. Spectral counts were used for relative quantification of MSP-MS results.

iii. Proteomic Analysis of Cyst Fluid Samples

Prior to proteomic analysis, abundant serum proteins were depleted from cyst fluid samples to improve detection of low abundance proteases. First, 5 μL of each cyst fluid sample was added to columns containing resin slurry for immunodepletion of the top 12 most abundant serum proteins (Thermo Fisher, 85164). Samples were incubated with the resin for 1 hour at room temperature. Columns were then placed in collection tubes and centrifuged for two minutes at 1000 g to collect unbound proteins. Protein concentration of the eluate was then determined by BCA assay (Thermo Fisher, 23225).

Serum depleted cyst fluid samples were then processed for proteomic analysis using a standard protocol. Briefly, 5 μg of cyst fluid protein was denatured in 6 mol/L urea, disulfide bonds were reduced with 10 mmol/L DTT, and free thiols were then alkylated with 12.5 mmol/L iodoacetamide. The urea concentration was diluted to 2 mol/L using 25 mmol/L ammonium bicarbonate and 100 ng of trypsin was added for 16 hours at 37° C. Following trypsinization, the sample was desalted using a C18 tip (Rainin), dried, and resuspended in 0.1% formic acid. One fifth of the cyst fluid sample was then injected onto an Orbitrap Fusion Lumos Mass Spectrometer (Thermo Fisher) coupled to an UPLC System (Waters). A 240 minute linear gradient from 2%-30% acetonitrile was used for peptide separation with a flow rate of 300 nL/min. Survey scans were recorded over a 375-1500 m/z range and the 20 most intense precursor ions from each survey scan were fragmented by high-energy collision dissociation (HCD).

Peak lists were generated from MS/MS data using an in-house software called PAVA and searched in Protein Prospector v. 5.10.0. Peak lists were searched against all human protein sequences in the SwissProt database (downloaded Nov. 1, 2017). This database was concatenated with a fully randomized set of entries to estimate the false discovery rate (FDR). For database searches, peptides sequences were matched to tryptic peptides with up to two missed cleavages. Carbamidomethylation of cysteine residues was used as a constant modification and variable modifications included oxidation of methionine, N-terminal pyroglutamate from glutamine, N-terminal acetylation, and loss of N-terminal methionine. The mass accuracy tolerance was set to 20 ppm for precursor ions and 30 ppm for fragment ions. An FDR of less than 1% was used for all searches. Raw mass spectrometry data files and peak list files have been deposited with accession number MSV000083051 at ProteoSAFE (http://massive.ucsd.edu); while under review the data may be accessed with the password “a.”

iv. Parallel Reaction Monitoring of Cyst Fluid Proteases

To select peptides for parallel reaction monitoring (PRM) assays, we initially performed shotgun proteomic analysis of recombinant TPP1, gastricsin, and cathepsin E (R&D Systems). First, 10 ng of recombinant protease was denatured, reduced, alkylated, and digested. Mass spectrometry data was then collected using the same system, method, and search parameters as described above for analysis of cyst fluid proteins. From the shotgun proteomic results, we selected two of the identified peptides for TPP1, gastricsin, and cathepsin E for inclusion in PRM assays (Table 5 below). Peptides were prioritized based on whether they were from the mature forms of the proteases and if they had been previously used in targeted proteomics assays (Kusebauch et al., 2016).

For PRM assays, 5 μL of cyst fluid protein was serum depleted as described above. The UPLC System was also operated using the same parameters. The MS acquisition method consisted of a full MS1 scan event followed by six targeted MS/MS scans for the peptides from TPP1, gastricsin, and cathepsin E. A 0.8 Da mass window was used for precursor ion isolation. The MS1 scan was performed at a resolving power of 120,000 while the MS/MS scans were performed at a resolving power of 30,000.

Relative quantitation of gastricsin and cathepsin E peptides was performed using the Skyline software package. Quantitation was based on the area under the curve of the eight most intense transitions for each peptide. To correct for potential differences in protein loading between runs, peak areas were normalized by the median peak area of all fragmented ions from shotgun proteomic analysis carried out using the same sample. The average area under the curve of the two peptides from each protease was then used to estimate the protein abundance in a given cyst fluid sample.

v. Peptide Synthesis

Solid-phase synthesis of internally quenched fluorescent substrates was carried out using standard Fmoc chemistry on a Syro II automated peptide synthesizer (Biotage). The peptides contain a C-terminal Fmoc-Lys(dinitrophenol) (Anaspec, A23856) and a Lys-(7-methoxcoumarin-4-acetic acid)-OH (EMD Millipore, 852095) in either the P3, P2, or P1 position. Amino acids were coupled to Wang resin preloaded with the Fmoc-Lys(dinitrophenol). The following conditions were used for coupling of Fmoc protected amino acids: 5 equivalents of amino acid, 4.9 equivalents of HCTU, and 20 equivalents of N-methylmorpholine. Peptides were cleaved from the resin by treatment with a solution of 95% trifluoroacetic acid, 2.5% Triisopropylsilane, and 2.5% water for 1 hour. Peptides were precipitated in cold diethyl ether and dried. Crude peptides were purified by HPLC and the chemical composition was confirmed through LC-MS analysis.

vi. TPP1 Activity Analysis Using Internally Quenched Fluorescent Substrates

Fluorescence-based TPP1 activity assays were performed in triplicate in black, round-bottom 384-well plates. A final volume of 15 μL of pH 4.5 acetate buffer was used for all assays. The pH of 4.5 was selected on the basis of promoting TPP1 activity (Tian et al., 2006). For analysis of TPP1 activity in cyst fluid samples, 2 μmol/L of pepstatin was included in the acetate buffer to inhibit residual aspartyl protease activity. Substrate concentration was 20 μmol/L unless otherwise stated. Recombinant TPP1 activity was assessed at 2 μmol/L, except for when determining the limit of detection when concentrations down to 1.5 pmol/L were assessed. Cyst fluid assays were carried out using 0.75 μL of sample per well. Substrate cleavage was monitored over 1 hour with a Synergy HT Plate Reader (Biotek) using excitation and emission wavelengths of 328 nm and 393 nm, respectively. Activity of cyst fluid samples and recombinant TPP1 is expressed as the initial velocity of substrate hydrolysis in relative fluorescent units per second (RFU/sec).

vii. Statistical Testing and Data Analysis

Two-tailed t-tests or two-way ANOVAs were used for assessing differences in protease activity between cysts. The specific test is indicated in text. All mass spectrometry data was log 2 transformed prior to statistical testing. Logistic regression models were employed for cyst prediction and generating receiver operating characteristic (ROC) curves. Youden's J statistic was used for identifying the optimal activity cutoff for assessing sensitivity and specificity of markers. RStudio was used to generate heatmaps, volcano plots, Venn diagrams, and ROC curves. GraphPad Prism was used for scatter plots, bar charts, and to fit kinetic data. Protease substrate specificity was visualized using iceLogo software ((Colaert et al., 2009). Gene Ontology (http://geneontology.org/) was used for annotating proteases from proteomics data.

2. Results

i. Aminopeptidase Activity is Enhanced in Mucinous Cysts

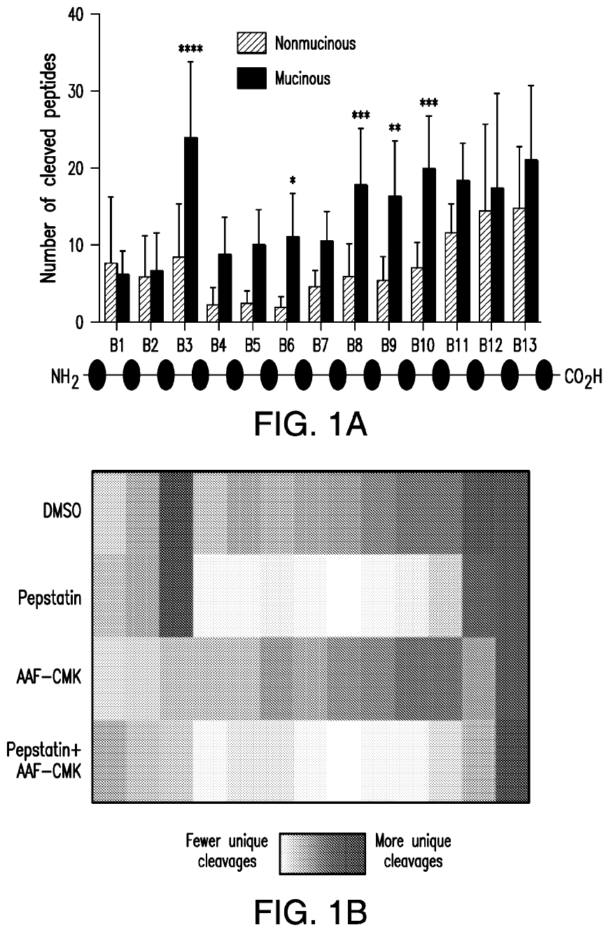

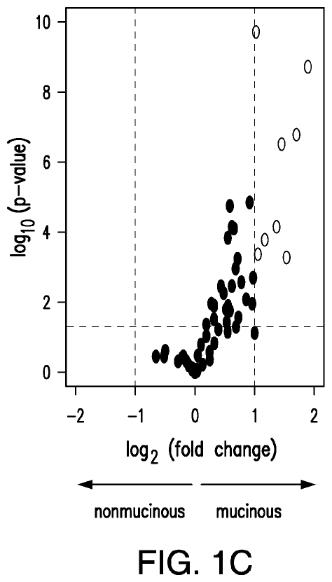

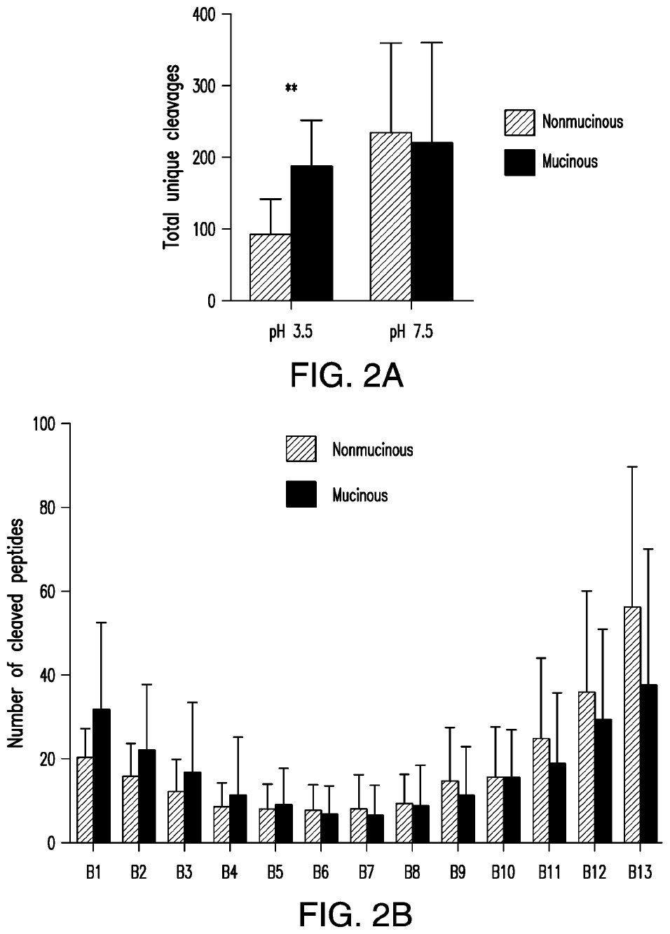

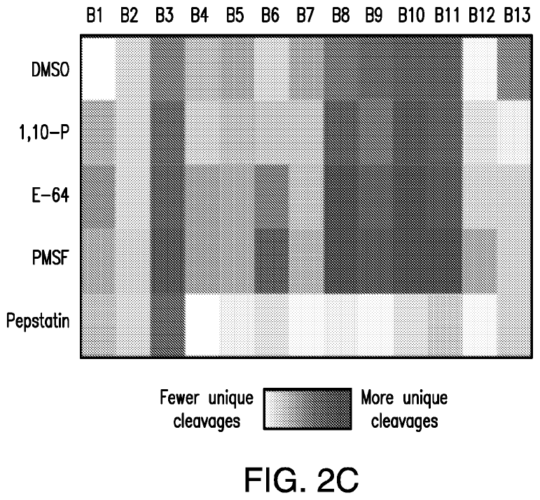

We previously used our MSP-MS assay to assess global proteolytic activity in fluid from 23 pancreatic cysts (Ivry et al., 2017). In the present study, we analyzed an additional 12 mucinous cysts using our MSP-MS assay. We performed the assay under both acidic conditions and at neutral pH. In line with our previous results, mucinous cysts displayed increased proteolytic activity under acidic conditions with over a 2-fold increase in the average number of detected peptide cleavages relative to nonmucinous cysts (FIG. 2A). In contrast, both cyst types cleaved similar numbers of peptides at neutral pH. We then analyzed which amide bond along the 14-mers from the 228 member peptide library used in MSP-MS was cleaved most frequently. We did not observe any significant differences at neutral pH (FIG. 2B). However, under acidic conditions, a number of amide bonds were cleaved to a greater extent in mucinous cysts and we were particularly interested to observe that cleavage after the third amino acid from the N-terminus was the most significantly enriched (FIG. 1A). This tri-amino peptidase activity was not inhibited by pepstatin (FIG. 1B and FIG. 2C), indicating that it was not driven by the pepstatin-sensitive aspartyl proteases identified previously. Pepstatin did decrease the number of detected peptide cleavages at the other positions that were enriched in mucinous cysts at acidic pH (B6, B8, B9, B10). Treatment with other broad-spectrum protease inhibitors also failed to impact the observed tri-amino peptidase activity (FIG. 2C), however, a tripeptide chloromethylketone inhibitor almost completely ablated this (FIG. 1B). Halomethylketone-based inhibitors are generally used to target cysteine and serine proteases, providing further evidence that non-aspartyl, protease activity is increased in mucinous cysts (Sanman & Bogyo, 2014).

We also analyzed all 93 detected tri-amino peptidase cleavage events to determine if individual tri-aminopeptidase cleavages were enriched in mucinous cysts (FIG. 1C). Indeed, nine unique events met our selectivity criteria for differentiating mucinous from nonmucinous cysts (+/−log 2(mucinous/nonmucinous), P<0.05). Collectively, these results suggest that non-aspartyl, tri-amino peptidase activity is increased in mucinous cysts and specific peptide cleavages might serve as a biomarker.

ii. Proteomic Analysis Identifies Increased Levels of TPP1 in Mucinous Pancreatic Cysts

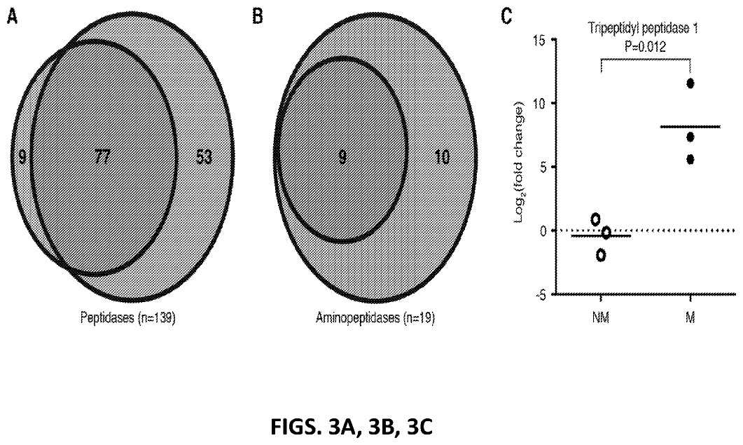

We next sought to identify the specific protease responsible for the increased tri-amino peptidase activity. In our previous study, we performed shotgun proteomic analysis of several cyst fluid samples and identified three aminopeptidases. However, all were metallopeptidases and, based on our inhibitor sensitivity data (FIG. 2C), are likely not driving the observed tri-amino peptidase activity. To increase the likelihood of identifying the target aminopeptidase here, we first performed a depletion step to remove the abundant serum proteins that are commonly detected in pancreatic cyst fluid. We then performed proteomic analysis and detected a total of 1,632 proteins between six cyst fluid samples. Gene ontology analysis revealed that 139 of these proteins are proteases, including 19 aminopeptidases (Table 4). Fifty-three of the identified proteases were only detected in mucinous cysts, while nine were selectively present in nonmucinous cysts (FIG. 3A). As expected, gastricsin and cathepsin E were only identified in mucinous cysts. The majority of aminopeptidases were also only present in mucinous cysts and none were exclusively found in nonmucinous cysts (FIG. 3B and Table 4).

TABLE 4
Peptidases and aminopeptidases identified in cyst fluid samples. All identified proteins and that were
annotated as peptidases or aminopeptidases through GO analysis. The number of unique peptides for all
detected peptidases in the nonmucinous (NM, n = 3) and mucinous cyst (M, n = 3) are reported.
For the identified aminopeptidases, the catalytic class is also reported. This table is an excerpt
from a larger table of identified enzymes and shows only those serine pro-teases identified in the study.

| | Accession | | | | Catalytic | Number Of Unique Peptides | | | | | |
|---|---|---|---|---|---|---|---|---|---|---|---|
| Rank | Number | UniProt ID | Gene | Protein Name | Class | NM1 | NM2 | NM3 | M1 | M2 | M3 |
| 473 | P29144 | TPP2_HUMAN | TPP2 | Tripeptidyl-peptidase 2 | Serine | | | | | 2 | 11 |
| 1002 | P42785 | PCP_HUMAN | PRCP | Lysosomal Pro-X | Serine | | | | 4 | | 1 |
| | | | | carboxypeptidase | | | | | | | |
| 1129 | Q9UHL4 | DPP2_HUMAN | DPP7 | Dipeptidyl peptidase 2 | Serine | 1 | | | 2 | 2 | |
| 1354 | P03951 | FA11_HUMAN | F11 | Coagulation factor XI | Serine | 1 | 2 | 1 | 1 | 2 | |
| 1452 | O14773 | TPP1_HUMAN | TPP1 | Tripeptidyl-peptidase 1 | Serine | | | | 1 | 1 | 1 |

To determine which of the pepstatin-insensitive aminopeptidases was responsible for the increased tri-amino peptidase activity we considered the following features: catalytic class, pH optimum, and which type of cyst they were detected in (Table 4). Only seven of the identified aminopeptidases were serine or cysteine proteases, which could be targeted by the halomethylketone-based inhibitor (FIG. 1B). Of these, only TPP1 and TPP2 are reported to be inhibited by the specific tripeptide chloromethylketone that we used (Gavioli et al., 2001; Lin et al., 2001). However, TPP2 is most active at neutral pH, whereas TPP1, which is normally localized to the lysosome, is known to be active under the acidic conditions in which we observed increased tri-aminopeptidase activity (Tian et al., 2006). TPP1 was also only detected in fluid from mucinous cysts, providing further evidence that it is driving the enhanced activity.

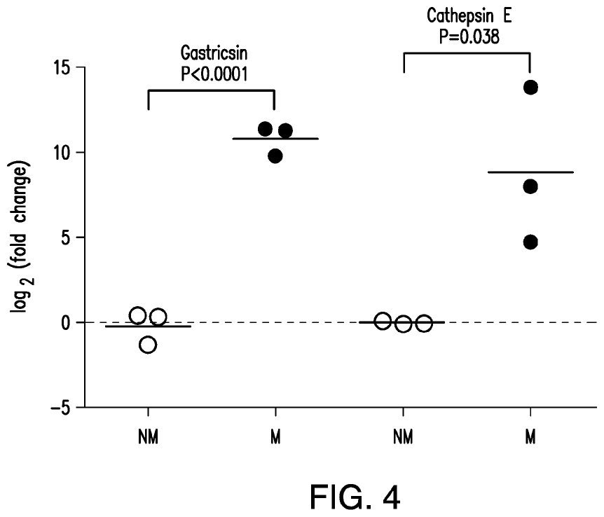

In order to confirm TPP1's increased abundance in mucinous cysts, we developed a PRM assay for relative quantitation of this protease through mass spectrometry. PRM enables the highly sensitive, analysis of targeted peptides for relative quantitation of proteins of interest. We also developed PRM assays for gastricsin and cathepsin E in order to compare the relative fold change in abundance for all the proteases we identified with increased activity in mucinous cysts. Our final PRM assay targeted two peptides per protease for a total of six peptides (Table 5). As expected based on our activity and shotgun proteomics data, TPP1 displayed significantly increased abundance in mucinous cysts (FIG. 3C). The log₂ (fold change) in abundance for TPP1 was 8.2, while for gastricsin and cathepsin E it was 11.6 and 9.5, respectively (FIG. 4).

TABLE 5
Analysis of TPP1, gastricsin, and cathepsin E relative abundance by PRM. TPP1,
gastricsin, and cathepsin E abundance were analyzed with a PRM assay in nonmucinous (NM,
n = 3) and mucinous cysts (M, n = 3). Reported peak areas are based on the 8 most abundant
transitions for each peptide.

| | Accession | | | | Peak Area | | | | | |
|---|---|---|---|---|---|---|---|---|---|---|
| Protein | Number | Peptide | m/z | z | NM1 | NM2 | NM3 | M1 | M2 | M3 |
| Cathepsin E | P14091 | QFYSVFDR | 531.2562 | 2 | 2606 | 11146 | 13246 | 239940247 | 204468 | 1828912 |
| Cathepsin E | P14091 | SQLSEFWK | 512.7585 | 2 | 4301 | 4068 | 3415 | 235387329 | 70887 | 4299258 |
| Gastricsin | P20142 | SYYSVYDLGNNR | 725.8335 | 2 | 1554 | 3576 | 3630 | 13785954 | 11531615 | 23011535 |
| Gastricsin | P20142 | GLLGEFLR | 452.7662 | 2 | 9996 | 25918 | 6194 | 28137132 | 24801435 | 64446224 |
| Tripeptidyl- | O14773 | LYQQHGAGLFDVTR | 535.6108 | 3 | 1391 | 4264 | 14085 | 12396244 | 817414 | 176277 |
| peptidase | | | | | | | | | | |
| Tripeptidyl- | O14773 | LFGGNFAHQASVAR | 492.2565 | 3 | 1150 | 4447 | 3669 | 16709112 | 752909 | 288863 |
| peptidase | | | | | | | | | | |

iii. TPP1 Activity Is Increased in Mucinous Cysts

Due to the increased abundance of TPP1 observed through targeted proteomic analysis, we decided to further pursue this protease as a putative biomarker for pancreatic cysts. To this end, we wanted to develop a simple, fluorescent assay for activity analysis in cyst fluid. Several fluorescent substrates have been reported for TPP1, however, we decided to leverage our MSP-MS assay to identify novel substrate sequences that might have improved turnover rates and improve the sensitivity for protease detection in clinical samples (Kondo et al., 2016; Tian et al., 2006). MSP-MS analysis of recombinant TPP1 confirmed that this protease readily accommodates hydrophobic amino acids in the P1 and P1′ positions, which flank the cleavage site (FIG. 5A). The P1 position also showed a slight preference for glutamine and aspartic acid. We then selected three TPP1 substrates from our MSP-MS library and incorporated the P3 to P4′ sequences into internally quenched fluorescent peptides. These substrates were selected based on several features. First, we identified substrates that showed time-dependent cleavage by recombinant TPP1 in our MSP-MS assay (FIG. 6A). Substrates also needed to generally follow the P1 and P1′ specificity of TPP1. We then prioritized substrates whose cleavage differentiated mucinous from nonmucinous cysts in our initial low pH MSP-MS analysis of cyst fluid and are likely indicative of TPP1 activity (FIG. 5B and FIG. 6B).

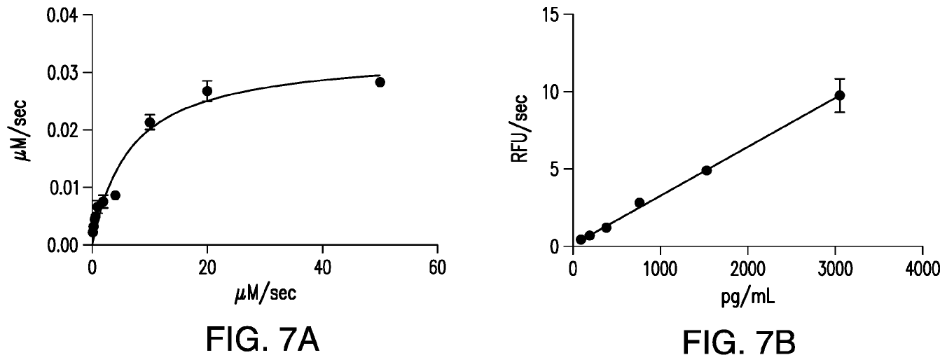

For synthesis of internally quenched substrates containing the selected P3 to P4′ sequences, we wanted to maintain the free N-terminal amine for recognition by TPP1. Therefore, the quencher was appended to the C-terminus while the fluorophore was conjugated to the side-chain amine of a lysine residue. We then tested which position on the nonprime-side of the scissile bond best accommodated this lysine-fluorophore (FIG. 5C and FIG. 6C). For our most rapidly cleaved substrate, this was the P1 position where the bulky lysine-fluorophore replaced a tryptophan residue. This substrate is cleaved with a kcat/Km of 2.5 μmol⁻¹s⁻¹, which is approximately two orders of magnitude faster than what is typically reported for the most commonly used substrate and we could use this to quantitate as little as 95 pg/mL of TPP1 (FIG. 7A and FIG. 7B) (Kondo et al., 2016; Tian et al., 2006).

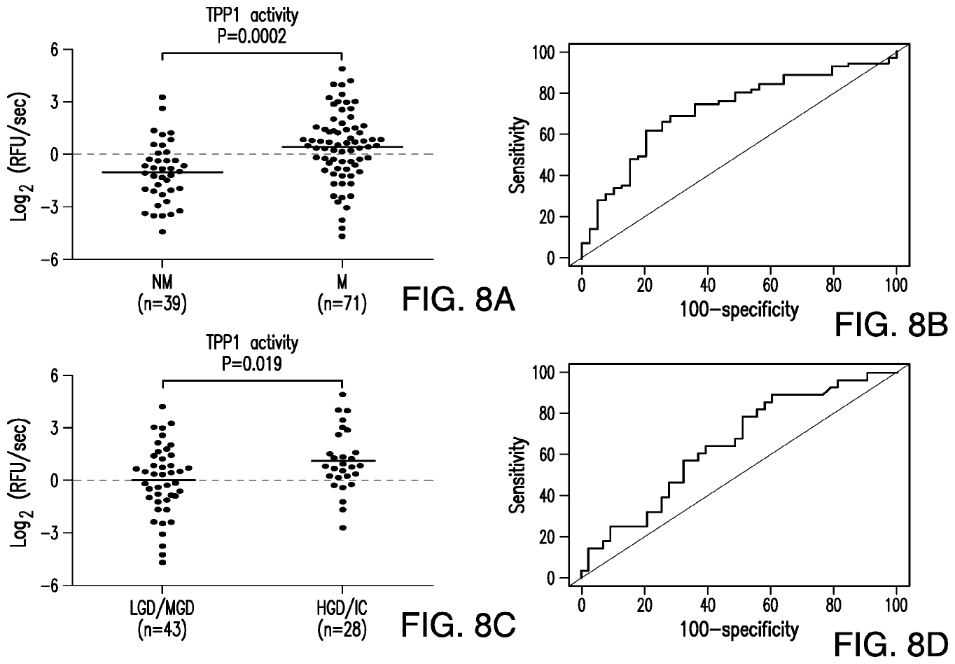

We next used this substrate to directly assess the levels of TPP1 activity in 110 cyst fluid samples, including the 35 samples that were previously analyzed by MSP-MS. Cleavage of our TPP1 substrate was increased approximately 3-fold in mucinous cysts relative to nonmucinous cysts (FIG. 8A). However, 27 mucinous cysts cleaved the TPP1 substrate at a lower rate than what was on average observed in nonmucinous cysts. Analysis of the receiver operating characteristic (ROC) curve for TPP1 activity revealed an area under the curve (AUC) of 0.72. At the optimal cutoff of 1.1 RFU/sec, the sensitivity and specificity for TPP1 activity analysis was 62% and 80%, respectively. This performance is comparable to what is generally reported for CEA (Scheiman et al., 2015), although within our cohort CEA at the standard cutoff of 192 ng/mL demonstrated a sensitivity of 65% and specificity of 94% (Ivry et al., 2017). Addition of TPP1 activity analysis to gastricsin, which we previously showed to be highly accurate for differentiating mucinous from nonmucinous cysts, did not lead to improved diagnostic performance of this marker (Table 6).

TABLE 6
Diagnostic performance of TPP1 activity analysis in 110 cyst
fluid sample. AUC of TPP1, gastricsin, and a combination of
the two biomarkers. For the biomarker combination, 10-fold
cross-validation was performed to assess model AUC. TPP1: Tripeptidyl-
peptidase 1; CTSE: cathepsin E; PGC: gastricsin.

| Markers | AUC | Sensitivity (%) | Specificity (%) |
|---|---|---|---|
| TPP1 | 0.721 | 62 | 79.5 |
| CTSE | 0.828 | 70.4 | 92.3 |
| PGC | 0.979 | 93 | 100 |
| TPP1 + CTSE | 0.849 | 74.6 | 87.2 |
| TPP1 + PGC | 0.981 | 93 | 100 |
| CTSE + PGC | 0.987 | 94.4 | 100 |
| TPP1 + CTSE + PGC | 0.987 | 94.4 | 100 |

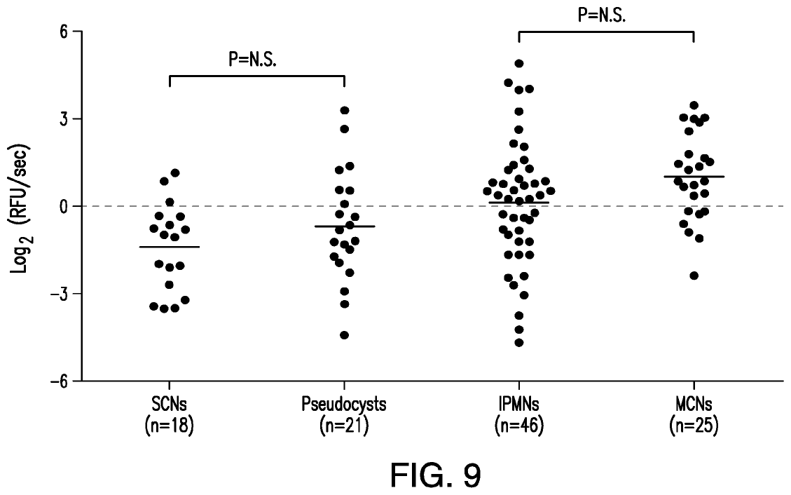

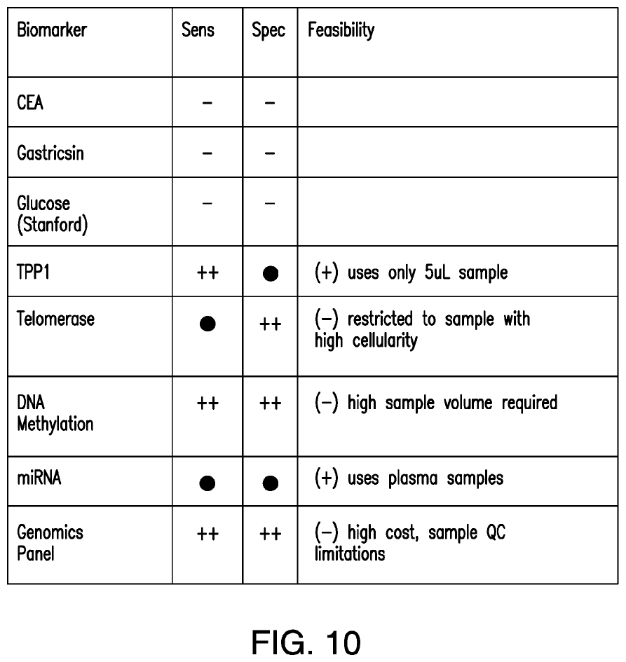

We also analyzed whether TPP1 activity differentiated the different types of mucinous and nonmucinous cysts (FIG. 9). SCNs generally had the lowest levels of TPP1 activity, although this was not significant relative to pseudocysts, which where were the other type of nonmucinous cyst included in this study. MCNs tended to have the highest TPP1 activity, however, this was not significant relative to IPMNs.

We next examined whether TPP1 activity levels were associated with the degree of dysplasia within a mucinous cyst. As mentioned previously, this is a critical distinction, as only cysts with HGD/IC generally need to be resected. Mucinous cysts with HGD/IC had a nearly 2-fold increase in TPP1 activity relative to cysts with LGD (FIG. 8C). The ROC curve for TPP1 activity displayed an AUC of 0.65 with a sensitivity of 89% and specificity of 40% (FIG. 8D). This performance is comparable to the ability of consensus guidelines to identify malignant mucinous cysts (Goh et al., 2014; Hsiao et al., 2016; Kaimakliotis et al., 2015).

3. Discussion

More than 80% of patients with newly diagnosed pancreatic cancer present with advanced disease, where surgical removal is no longer an option (Rahib, Fleshman, Matrisian, & Berlin, 2016). Early detection of pancreatic cysts presents a unique opportunity for curative resection of this highly lethal disease. Unfortunately, current management guidelines and diagnostic tools are unable to definitively differentiate the cysts that are most likely to progress to pancreatic cancer. Up to two thirds of resected pancreatic cysts are benign or only contain LGD with little risk of histological progression, thus exposing these patients to unnecessary risk of surgical morbidity and mortality (Correa-Gallego et al., 2010; Hsiao et al., 2016; Parra-herran et al., 2010; Scheiman et al., 2015; Valsangkar et al., 2012).

In a previous study, we identified gastricsin and cathepsin E as promising, cyst fluid-based biomarkers for differentiating mucinous from nonmucinous pancreatic cysts (Ivry et al., 2017). Here, we applied MSP-MS combined with shotgun and targeted quantitative proteomic analysis to search for additional proteases that had both increased activity in mucinous cysts and could differentiate cysts based on their degree of dysplasia. We determined that activity of the aminopeptidase TPP1 is significantly increased in mucinous cysts, but does not differentiate these lesions from nonmucinous cysts as well as gastricsin, cathepsin E, or CEA. Within mucinous cysts, TPP1 activity is most highly elevated when HGD/IC is present. As a stand-alone marker, TPP1 activity had modest 89% sensitivity and 40% specificity for differentiating mucinous cysts with HGD/IC from those with LGD. However, this diagnostic performance of this simple assay compares favorably to the clinical and radiologic features that are commonly assessed with current management guidelines and could be particularly useful when specific features, such as cytology, fail to provide a clear diagnosis (Scheiman et al., 2015). TPP1 may improve the diagnostic performance of other promising cyst fluid biomarkers that are emerging when used in combination (Hata et al., 2017; Hata et al., 2016; Singhi et al., 2017; Springer et al., 2015). For example, a sequential diagnostic strategy could be employed where gastricsin and cathepsin E are used to determine if a pancreatic cyst is mucinous and then TPP1 in combination with other markers is used to identify which mucinous cysts contain HGD/IC. This will be a primary focus of future work investigating TPP1 activity in pancreatic cysts.

TPP1 is best studied in the context of the childhood neurodegenerative disease, classic late-infantile form of neuronal ceroid lipofuscinoses (CLN2) and has only rarely been associated with tumorigenesis (Golabek & Kida, 2006). CLN2 is a lysosomal storage disease that is driven by mutations in TPP1 that lead to reduced protease activity (Sleat et al., 1997). Like many lysosomal hydrolases, TPP1 is most active under acidic conditions and its tri-aminopeptidase activity has a pH optimum of 4.5 (Tian et al., 2006). In line with our previous paper, our results here continue to point to increased abundance of acid-activated, lysosomal proteases in mucinous pancreatic cysts. This observation may be related to the increased reliance on lysosomal function for nutrient scavenging that is observed with pancreatic intraepithelial neoplasia-derived pancreatic cancer (Bai et al., 2015; Perera et al., 2015; Perera & Bardeesy, 2015). Many acid-activated, lysosomal proteases exhibit increased expression in these tumors and are secreted into the surrounding microenvironment. The abundance of lysosomal proteases in fluid from mucinous cysts suggests that these enzymes are also important for maintenance and growth in cyst-derived pancreatic cancer. In further support of this, several other lysosomal proteases, such as cathepsins B, L, and S, were detected through proteomic analysis of the cyst fluid samples included in this study. Additional studies will seek to determine if analysis of these acid-activated proteases improves diagnostic performance when combined with our current markers.

In conclusion, we demonstrate here that acid-activated aminopeptidase activity is elevated in mucinous pancreatic cysts and this is driven by the lysosomal protease TPP1. Activity analysis of TPP1 is a promising biomarker for differentiating nonmucinous from mucinous cysts and may help address the most critical clinical challenge of identifying mucinous cysts with HGD/IC. Validation of these results has the potential to assist clinical decision making for pancreatic cysts to help ensure appropriate treatment of these challenging precursor lesions of pancreatic cancer.

### Example 2: Validation of a Multimodal Protease-Based Diagnostic for Pancreatic Cysts

Our work has demonstrated that protease activity is a promising new biomarker for differentiating pancreatic cysts based on their likelihood of malignant transformation. We have already shown diagnostic utility for our activity-based biomarkers in a cohort of 110 patient samples and our immediate next step will be validating diagnostic performance using a blinded patient cohort. We are currently working with a consortium of pancreatic cancer centers to assemble a cohort of between 200-400 patient cyst fluid samples. Prior to analysis of our validation cohort, we will need to establish the optimal cutoff for each activity-marker, determine our limit of detection, standardize sample treatment, and perform various other analytical validations steps.

In parallel, we also plan to develop a colorimetric, chemiluminescent, and enzyme-linked immunosorbent assay (ELISA) for each protease target. Colorimetric assays, although not as sensitive as fluorescence, are simple to automate and can be readily adopted into a clinical laboratory setting. ELISAs and chemiluminescence are both highly sensitive and might enable a lower limit of detection, in turn leading to improved diagnostic performance. ELISAs also have the potential for increased selectivity as antibodies tend to be much more specific than peptide substrates for assessment of a single analyte. We will assess each assay format within our training cohort of 110 cyst fluid samples to prioritize which to carry forward to validation. Assay type performance is generally dependent on the marker being targeted and it is possible that multiple formats will be necessary for optimal diagnostic performance.

Our results show that gastricsin and cathepsin E accurately differentiate mucinous from nonmucinous cysts, while TPP1 is better for distinguishing mucinous cysts based on their grade of dysplasia. These are both critical distinctions that need to be made to ensure appropriate management of pancreatic cysts. Therefore, we anticipate a sequential diagnostic strategy for clinical use of our protease-based biomarkers. First, mucinous cysts will be identified using gastricsin and cathepsin E analysis. TPP1 assessment will then identify those mucinous cysts with HGD/IC that should be resected. Collectively, these results suggest that a panel of biomarkers, including the protease-based markers identified here, will be required for the appropriate management of pancreatic cysts.

### Example 3: Protease-Activated Chemotherapeutics for Treatment of Pancreatic Cancer

Through this work we have identified several proteases whose expression is primarily associated with PDAC and its precursor lesions. We have primarily leveraged the activity of these proteases for the development of diagnostics. We are now beginning to investigate whether PDAC-associated proteases could be used to activate therapeutics within the tumor microenvironment for cancer treatment. More specifically, we plan to synthesize chemotherapeutics that are conjugated to a masking peptide that blocks either cellular permeability or target engagement. The peptide mask is stable in circulation, but can be cleaved by a protease within the tumor microenvironment to release the active drug. Similar strategies have been previously employed for both small molecules and biologics in cancer (Wu et al., 2006; Denmeade et al., 2012; Desnoyers et al., 2013). These efforts have shown promise for improving the therapeutic window of their parent agents. However, most efforts have been limited to targeting the mask to a single tumor-associated protease. Through MSP-MS, we have obtained a more global view of PDAC-associated proteolysis and believe this will allow us to design masking peptides that are highly selective for multiple proteases within the tumor microenvironment. In combination with the diagnostic work proposed above, we believe these efforts may lead to highly personalized treatment regimens for pancreatic cancer patients.

### Example 4: Pancreatic Cyst Risk Stratification for Early Detection of Pancreatic Cancer Using Multi-Analyte Functional Cyst Fluid Biomarkers

We will validate an enzymatic activity-based pancreatic cyst biomarker approach and further develop and prospectively validate its use in a proposed two-tier classification system designed to discriminate clinically relevant from non-relevant pancreatic cysts in a well annotated and generalizable clinical cohort. In the first experiment, we will validate our published classifiers of mucinous and nonmucinous cysts (Tier 1) and of LGD and HGD/I among mucinous cysts (Tier 2), using resected cyst fluid samples from patients enrolled across our healthcare center. In the second experiment, we will combine the two Tiers into a classifier for risk stratification and determine if clinical features from the Fukuoka Guidelines further improve its performance. In the third broad-based experiment, we will prospectively estimate the impact on clinical decision-making and outcomes that would be expected if the two-tier classifier from Aim 2 were integrated into the Fukuoka Guidelines.

i. Rationale

Use a clinically relevant multi-center patient cohort (est. n=132) to validate and further develop our published functional cyst fluid biomarkers. We have evaluated the diagnostic performance of each of these activity tests in a cohort of 110 cyst fluid samples, and showed that gastricsin and TPP1 together have favorable performance compared to current standard of care cyst fluid-based assay (CEA) for distinguishing mucinous from non-mucinous cysts (Tier 1 component of the test). We will use previously untested cyst fluid samples obtained from resected, pathologically confirmed, pancreatic cysts in biorepositories across the UC-PCC and collected during Year 1 of the funding period. We will independently validate the performance of gastricsin, alone (1A) and in combination with TPP1, glucose, and the current standard CEA (1B), in the detection of mucinous (vs. non-mucinous) cysts (Tier 1). Secondly, we will independently validate the performance of TPP1, alone (1A) and in combination with gastricsin, CEA, and other promising protease analytes (1B), in the detection of HGD/invasive cancer (HGD/I) (Tier 2) (est. n=102 mucinous). We will rigorously evaluate the laboratory performance of our protease assays (1C).

ii. Selection of Performance Targets

The final goal of the diagnostic test for the Tier 1 classification of mucinous cysts should prioritize NPV (i.e. high sensitivity to identify any mucinous cysts), so that patients with non-mucinous cysts can safely be removed from surveillance or surgical consideration. Currently, the only routinely used diagnostic study for identification of mucinous cysts is CEA with a sensitivity of 63-73%, and specificity of 65-84% using a cutoff of 192 mg/dl. Our goal would be to improve the sensitivity of this assay, alone or in combination with other analytes to >90%, while maintaining high specificity. For the Tier 2 assay, there are no cyst fluid analytes in routine clinical use that reliably differentiate histologic grade, and while cytology has high specificity, sensitivity estimates range from 29-48%. Hence we estimate that a sensitivity >70% at acceptable specificity for detection of HGD/Invasive cancer would be clinically important, either alone or in combination with cytology. The laboratory performance targets will be set in collaboration with the BTT Core, but may include sample protease stability at room temperature for 2-24 hours, at 4° C. for 72 hours, and after 1, 2, and 3 sequential freeze/thaws cycles.

In the first clinical experiment is to independently validate our published classifier of mucinous vs non-mucinous cysts, which uses the proteolytic signature of the cyst fluid biomarker gastricsin in a functional high-throughput assay; and Tier 2 is to independently validate the performance of our published classifier of high vs low grade dysplasia among mucinous cysts, which uses the cyst fluid biomarker TPP1. We will determine if the addition of TPP1, glucose, and/or CEA will further improve performance of the published Tier 1 classifier and determine if the addition of gastricsin, CEA, or novel analytes will further improve performance of the Tier 2 classifier. We will comprehensively assess the laboratory performance of the enzymatic assays proposed for use in the final classifier.

In this second, forward-going experiment, we will use the same clinically relevant multi-center patient cohort (n=132) to develop a two-tier classifier for risk-stratification of pancreatic cysts, with an estimated sensitivity of 98% and specificity greater than 70% for identification of mucinous cysts with high-grade dysplasia or carcinoma.

iii. Rationale

In preparation for planned integration of our two-tier classifier into the Fukuoka Guidelines for use in prospective decision-making, we will evaluate the impact of clinical factors on the performance of each of the classifiers. Each of the 12 clinical data elements in the latest (2017) version of the Fukuoka Guidelines, collected from the same patients as in Aim 1, will be potentially incorporated into a multivariable classification rule, for Tier 1 and for Tier 2. Then the two Tiers will be combined into an overall classifier to identify mucinous lesions with HGD.

iv. Selection of Performance Targets

For Tier 1, as the Fukuoka Guidelines contain no clinical data elements to distinguish between mucinous and nonmucinous cysts, we anticipate the projected sensitivity, specificity will remain the same as stated in Aim 1. For Tier 2 of our classifier, we propose the inclusion of cyst fluid cytology will strengthen the NPV to a value greater than 80%. In prior publications, cytology, which is the only cyst fluid classifier for grade of dysplasia, was non diagnostic in 38% of samples. It will be important to determine how our Tier 2 classifier performs in this group of cytologically indeterminant samples.

In this example we will prospectively evaluate the ability of the two-tier multi-analyte classifier from the first set of experiments to improve the performance of the Fukuoka Guidelines for risk stratification of pancreatic cysts, in a blinded and rigorous validation study using a clinically relevant cohort of patients with pancreatic cysts.

v. Rationale

The retrospective validation and further development of the classifier using pathologically confirmed samples identified in previous examples and used resected specimens, and these are necessarily biased toward higher grade tumors. However, the planned integration of the two-tier classifier into clinical practice will occur using samples acquired during EUS, and most of these will be obtained during the initial diagnostic evaluation of a patient recently identified by CT/MRI with a pancreatic cyst, and in some cases, when a repeat EUS is done to further evaluate a cyst that has enlarged or developed other “worrisome features” per guidelines. The majority of these patients (approximately 70%) will not undergo surgery, and will be followed with CT/MRI every 6 months for 2 years then yearly for at least 5-10 years, thereby providing an opportunity to evaluate the “clinical biology” of the lesion. For the purposes of the study, we will classify a cyst as “indolent” if there is no development of “high risk stigmata” during 2 years of follow up, as previously described and used in the design of our current MCL U01 consortium.

vi. Selection of Performance Targets

The principle outcome is presence of HGD or pancreatic cancer. We will also count development of high-risk stigmata as a true positive, given that an estimated 25% of patients with high risk lesions will have HGD or pancreatic cancer within 10 years. Together, we term these ‘clinically actionable’ cysts. Thus, for the gold standard assessment, a true positive is defined in this study as a cystic lesion that is followed and develops high-risk stigmata, or a cyst that is eventually resected and is found to harbor HGD or invasive carcinoma. We will consider lesions that are not associated with development of HRS over two years as “indolent.” A gold standard negative is thus either: a lesion that is eventually resected and is either non-mucinous or low grade mucinous, or a patient who is followed for 2 years (without resection) without developing high-risk stigmata. Patients who have been followed for less than 2 years without becoming a “true positive” will be considered “censored” (i.e. undetermined outcome at the time of analysis). Using these definitions, we recognize and estimate that up to approximately of 5% of our gold standard ‘true negatives’ may be false negatives; for low-risk IPMNs (essentially those without high-risk stigmata) the estimated cumulative incidence of high-grade dysplasia or pancreatic cancer was 3.12% (95% CI, 1.12-5.90%) at 5 years, and 7.77% (95% CI, 4.09-12.39%) at 10 years.

We plan to prospectively validate our two-tier classifier developed by incorporating the promising analytes and the informative clinical features using cyst fluid samples obtained during diagnostic EUS-FNA. A strength is that this validation study will enroll patients with indeterminate cysts that undergo clinically-directed diagnostic EUS procedures for the purpose of further clarifying disease classification, the exact setting in which the diagnostic is planned to be used. Clinicians will be unaware of the results of the study during the period of follow up and we plan to enroll patients at all healthcare centers.

In aim 3 we will apply the risk stratification from the two-tier classifier developed through Aims 1 and 2 to a prospective patient cohort, which is followed for outcomes of interest. Importantly, treatment will be standard-of-care, and the treating team will be blinded to the results of the novel risk stratification, as it has not yet been validated. On initial evaluation, each patient will be assigned as mucinous or non-mucinous at Tier 1, and, among those called as mucinous, as high grade or not at Tier 2, for final assignment as actionable (high grade mucinous) or indolent (nonmucinous, or low grade mucinous), according to the classification method developed in Aim 2. Patients will then be followed for outcome. Sensitivity, specificity, positive and negative predictive values will be presented overall, and for important clinical and demographic subgroups. The novel classifier will also be compared to the results of the Fukouka Guidelines: the estimated net difference in the per capita number of actionable cysts which are missed, and in the per capita number of indolent cysts falsely identified as high risk, will be presented, along with 95% Agresti-Coull confidence intervals. For resected patients, it will also be possible to validate the Tier 1 and Tier 2 results independently. Cytology will be analyzed at each respective site using the standard classification scheme for low and high grade dysplasia and invasive carcinoma.

Five years into the study, we anticipate having at least 600 subjects with initial EUS and a full 2 years of follow-up (280 subjects/year×0.7 not resected yields about 200 per year, over the first 3 years of the grant). In addition, we anticipate having about 200 subjects with resection and final pathology (66 per year, over years 2, 3, 4 of the grant). Of resected subjects, we anticipate that about 50% will be true indolent cases. Of EUS subjects, we anticipate about 10% may represent true actionable lesions. Thus we anticipate approximately 160 true actionable lesions, and 740 indolent lesions. With these sample sizes, assuming final sensitivity of 95% and final specificity of 70%, the half width of the respective confidence intervals would be 3.5 percentage points and 3.3 percentage points. Thus, these sample sizes are appropriate to be informative. For positive and negative predictive values, note that we anticipate there would be 152 (0.95×160) true positives and 8 false negatives; and also 518 true negatives and 222 false positives. Thus the estimated PPV would be 60% (152/(152+222)) and the estimated NPV would be 98.5% (518/(518+8)). The half width for the associated confidence intervals would be around 3 percentage points and 1 percentage point, respectively.

vii. Cohort Creation and Cyst Fluid Collection

Patients will be identified from endoscopic or surgical procedural schedules, or from the gastroenterology, general surgery, or pancreatic cyst clinics at all participating sites and consented per standard IRB protocols. Clinical data elements will be obtained from electronic medical record reviews and in-person interviews, and entered into a HIPAA-compliant secure database involving the 5 participating sites to ensure uniform collection of relevant demographic, clinical, procedural, and specimen data. Ten cc of plasma and 10 cc serum will be collected at the time of IV insertion per standard of clinical care, or from venous or arterial catheters already in-place per our IRB protocol (see Human Subject's form). Blood will be biobanked for future relevant hypothesis testing and analyte discovery or validation. Serum laboratory values that are obtained per standard of clinical care often include HbA1C, and CA19-9. The cyst fluid CEA will be sent to a standard commercial CLIA-approved lab for standard of clinical care testing with subsequent results reporting in our database. A formal diagnostic EUS will be performed and the reports collected and entered into our RedCap database. Data to analyze the 12 clinical data elements in the Fukuoka Guidelines will also be routinely obtained and recorded. The CRCs will be trained to obtain the specific CDEs described in the Fukuoka Guidelines. We will evaluate the performance of the 2-tiered classifier as described above.

By enrolling this large cohort of patients, we can also begin to “fill in the gaps” in our understanding and develop hypotheses for future testing. For example, results of serial sampling from patients that undergo repeat EUS and cyst fluid sampling after radiographic change is detected may allow us to determine if radiographic change is associated with changes in proteolysis and disease progression. We will also determine the performance of the assays in atypical cyst types (cystic neuroendocrine tumors, solid pseudopapillary tumors, and lymphoepithelial cysts, among others) so we can inform clinicians about the expectations of our analytes if these tumors are being clinically considered.

### REFERENCES

Bai, G., et al. (2015). Exploring the Functional Disorder and Corresponding Key Transcription Factors in Intraductal Papillary Mucinous Neoplasms Progression. International Journal of Genomics, 2015.
Brugge, W. R., et al. (2004). Diagnosis of Pancreatic Cystic Neoplasms: A Report of the Cooperative Pancreatic Cyst Study. Gastroenterology, 126, 1330-1336.
Cao, Z., et al. (2013). Specific glycoforms of MUCSAC and endorepellin accurately distinguish mucinous from nonmucinous pancreatic cysts. Molecular & Cellular Proteomics, 12, 2724-34.
Carr, R. A., et al. (2017). Pancreatic cyst fluid glucose: Rapid, inexpensive, and accurate diagnosis of mucinous pancreatic cysts. Surgery, 163, 600-605.
Colaert, N., et al. (2009). Improved visualization of protein consensus sequences by iceLogo. Nature Methods, 6, 786-7.
Correa-Gallego, et al. (2010). Incidental pancreatic cysts: Do we really know what we are watching? Pancreatology, 10, 144-150.
Gavioli, R., et al. (2001). C-myc overexpression activates alternative pathways for intracellular proteolysis in lymphoma cells. Nature Cell Biology, 3, 283-288.
Goh, B. K. P., et al. (2014). Utility of the Sendai Consensus Guidelines for Branch-Duct Intraductal Papillary Mucinous Neoplasms: A Systematic Review. Journal of Gastrointestinal Surgery, 18, 1350-1357.
Golabek, A. A., & Kida, E. (2006). Tripeptidyl-peptidase I in health and disease. Biological Chemistry, 387(8), 1091-1099. http://doi.org/10.1515/BC.2006.135
Hata, T., et al. (2017). Predicting the grade of dysplasia of pancreatic cystic neoplasms using cyst fluid DNA methylation markers. Clinical Cancer Research, 23
Hata, T., et al. (2016). Cyst fluid telomerase activity predicts the histologic grade of cystic neoplasms of the pancreas. Clinical Cancer Research, 5141-5151.
Hsiao, C. Y., et al. (2016). Utility of the 2006 Sendai and 2012 Fukuoka guidelines for the management of intraductal papillary mucinous neoplasm of the pancreas: A single-center experience with 138 surgically treated patients. Medicine, 95.
Ivry, S. L., et al. (2017). Global protease activity profiling provides differential diagnosis of pancreatic cysts. Clinical Cancer Research, 16, 4865-74.
Kaimakliotis, P., et al. (2015). Sendai and Fukuoka Consensus Guidelines Identify Advanced Neoplasia in Patients With Suspected Mucinous Cystic Neoplasms of the Pancreas. Clinical Gastroenterology and Hepatology, 13, 1808-1815.
Ke, E., et al. (2009). Proteomic Analyses of Pancreatic Cyst Fluids. Pancreas, 38, 1-21.
Kondo, M. Y., et al. (2016). Analysis of catalytic properties of tripeptidyl peptidase i (TTP-I), a serine carboxyl lysosomal protease, and its detection in tissue extracts using selective FRET peptide substrate. Peptides, 76, 80-86.
Kusebauch, U., et al. (2016). Human SRMAtlas: A Resource of Targeted Assays to Quantify the Complete Human Proteome. Cell, 166, 766-778.
Lee, K. S., et al. (2010). Prevalence of incidental pancreatic cysts in the adult population on MR imaging. The American Journal of Gastroenterology, 105, 2079-84.
Lin, L., et al. (2001). The human CLN2 protein/tripeptidyl-peptidase I is a serine protease that autoactivates at acidic pH. Journal of Biological Chemistry, 276, 2249-2255.
Maker, A. V., et al. (2008). Cytology from pancreatic cysts has marginal utility in surgical decision-making. Annals of Surgical Oncology, 15, 3187-3192.
Matthaei, H., et al. (2012). miRNA biomarkers in cyst fluid augment the diagnosis and management of pancreatic cysts. Clinical Cancer Research, 18, 4713-4724.
Moris, M., et al. (2016). Association Between Advances in High-Resolution Cross-Section Imaging Technologies and Increase in Prevalence of Pancreatic Cysts From 2005 to 2014. Clinical Gastroenterology and Hepatology, 14, 585-593.
O'Donoghue, et al. (2012). Global identification of peptidase specificity by multiplex substrate profiling. Nature Methods, 9, 1095-1100.
Park, W. G., et al. (2011). Diagnostic Performance Of Cyst Fluid Carcinoembryonic Antigen And Amylase In Histologically Confirmed Pancreatic Cysts. Pancreas, 40, 42-45.
Parra-herran, C. E., et al. (2010). Cystic Lesions of the Pancreas: Clinical and Pathologic Review of Cases in a Five Year Period. Journal of the Pancreas, 11, 358-364.
Perera, R. M., & Bardeesy, N. (2015). Pancreatic cancer metabolism: Breaking it down to build it back up. Cancer Discovery, 5, 1247-1261.
Perera, R. M., et al. (2015). Transcriptional control of autophagy-lysosome function drives pancreatic cancer metabolism. Nature.
Rahib, L., et al. (2016). Evaluation of Pancreatic Cancer Clinical Trials and Benchmarks for Clinically Meaningful Future Trials. JAMA Oncology, 2, 1209.
Sanman, L. E., & Bogyo, M. (2014). Activity-based profiling of proteases. Annual Review of Biochemistry, 83, 249-73.
Scheiman, J. M., et al. (2015). American gastroenterological association technical review on the diagnosis and management of asymptomatic neoplastic pancreatic cysts. Gastroenterology, 148, 824-48.e22.
Singhi, A. D., et al. (2017). Preoperative next-generation sequencing of pancreatic cyst fluid is highly accurate in cyst classification and detection of advanced neoplasia. Gut.
Sinha, J., et al. (2016). A Gastric Glycoform of MUCSAC Is a Biomarker of Mucinous Cysts of the Pancreas. Plos One, 11, e0167070.
Sleat, D. E., et al. (1997). Association of Mutations in a Lysosomal Protein with Classical Late-Infantile Neuronal Ceroid Lipofuscinosis. Science, 277, 1802-1806.
Springer, S., et al. (2015). A Combination of Molecular Markers and Clinical Features Improve the Classification of Pancreatic Cysts. Gastroenterology, 149, 1501-1510.
Tanaka, M., et al. (2012). International consensus guidelines 2012 for the management of IPMN and MCN of the pancreas. Pancreatology, 12, 183-97.
Tian, Y., et al. (2006). Determination of the substrate specificity of tripeptidyl-peptidase I using combinatorial peptide libraries and development of improved fluorogenic substrates. Journal of Biological Chemistry, 281, 6559-6572.
Valsangkar, N. P., et al. (2012). 851 resected cystic tumors of the pancreas: A 33-year experience at the Massachusetts General Hospital. Surgery, 152, 4-12.
Waaij, L. A. Van Der, et al. (2005). Cyst fluid analysis in the differential diagnosis of pancreatic cystic lesions: a pooled analysis. Gastrointestinal Endoscopy, 62, 383-389.
Wang, J., et al. (2015). Next generation sequencing of pancreatic cyst fluid microRNAs from low grade-benign and high grade-invasive lesions. Cancer Letters, 356, 404-9.
Wu, J., et al. (2011). Whole-exome sequencing of neoplastic cysts of the pancreas reveals recurrent mutations in components of ubiquitin-dependent pathways. Proceedings of the National Academy of Sciences of the United States of America, 108, 21188-93.
Wu, J., et al. (2011). Recurrent GNAS mutations define an unexpected pathway for pancreatic cyst development. Science Translational Medicine, 3, 92ra66.
Zikos, T., et al. (2015). Cyst Fluid Glucose is Rapidly Feasible and Accurate in Diagnosing Mucinous Pancreatic Cysts. The American Journal of Gastroenterology, 110, 909-14.
W. Wu, et al., Targeting cell-impermeable prodrug activation to tumor microenvironment eradicates multiple drug-resistant neoplasms, Cancer Res. 66 (2006) 970-980.
S. R. Denmeade, et al., Engineering a Prostate-Specific Membrane Antigen-Activated Tumor Endothelial Cell Prodrug for Cancer Therapy, Sci. Trans!. Med. 4 (2012).
L. R. Desnoyers, et al., Tumor-specific activation of an EGFR-targeting probody enhances therapeutic index, Sci. Transl. Med. 5 (2013).
Ivry S. L., et al. The lysosomal aminopeptidase tripeptidyl peptidase 1 displays increased activity in malignant pancreatic cysts. Biological Chemistry. 2019; 400(12):1629-1638.

1. A method of diagnosing a subject with a malignant growth of the pancreas comprising:
detecting the presence, absence, and/or quantity of at least one serine protease or functional fragment thereof in a sample from the subject.

2. (canceled)

3. The method of claim 1, wherein the step of detecting is preceded by a step of acquiring the sample from the subject.

4. The method of claim 1 further comprising exposing a sample from a subject to at least one substrate of a serine protease or functional fragment thereof or at least one molecule capable of reacting with or binding to at least one serine protease or functional fragment thereof.

5. The method of claim 4 further comprising exposing a sample from a subject to at least one substrate of an aspartyl protease or functional fragment thereof or at least one molecule capable of reacting with or binding to at least one aspartyl protease or functional fragment thereof.

6. The method of claim 4, wherein the serine protease comprises at least about 70%, sequence identity to SEQ ID NO: 10 or is a variant thereof comprising at least about 70%, sequence identity to SEQ ID NO: 10.

7. The method of claim 5, wherein the at least one aspartyl protease is a cathepsin E comprising at least about 70%, sequence identity to SEQ ID NO: 11, or a gastricsin comprising at least about 70% sequence identity to SEQ ID NO: 12, or a variant thereof, or combinations thereof.

8. (canceled)

9. The method of claim 1, wherein the step of detecting the presence, absence, and/or quantity of at least one serine protease or functional fragment thereof in a sample comprises measuring the quantity of at least one serine protease or variant thereof by contacting the sample with one or a plurality of substrates specific for the at least one serine protease or variant thereof, for a time period sufficient to create a quantity of reaction product, and normalizing the quantity of the reaction product with a measurement of reaction product detected from a control sample.

10. (canceled)

11. The method of claim 9, further comprising correlating the amount of at least one serine protease, variant thereof in the sample to the amount of reaction product formed after the step of exposing the sample an enzymatically effective amount of substrate; and determining the probability or likelihood the subject has a malignant growth, relative to a measurement of the amount of serine protease or variant thereof in a control sample.

12. The method of any of claim 1, wherein the malignant growth is a mucinous or nonmucinous cyst.

13. The method of claim 1, wherein the sample is cystic fluid from a subject.

14. (canceled)

15. A method of diagnosing a subject with a mucinous cyst of the pancreas characterized by high dysplasia or invasive cancer comprising: detecting the presence, absence, and/or biologically significant quantity of a serine protease, TPP-1 or functional fragment or variant thereof in a sample from the subject.

16. The method of claim 15, wherein, before the step of detecting, the method further comprises the step of exposing the sample to an enzymatically effective amount of a substrate specific to TPP-1, or functional fragment or variant thereof for a time period sufficient to catalyze a reaction between the TPP-1, functional fragment of variant thereof and the substrate.

17. The method of claim 16 wherein the step of detecting the biologically significant quantity of TPP-1 or functional fragment or variant thereof comprises calculating a score corresponding to a normalized amount of TPP-1 or functional fragment or variant thereof in the sample based upon the amount of fragment of enzymatically effective amount of substrate detected as compared to the amount of fragment of enzymatically effective amount of substrate detected after exposure to the same amount of enzymatically effective amount of substrate exposed to a control sample.

18. The method of claim 17, wherein the quantity of fragment of substrate detected by the reaction is quantified by fluorescent probe, antibody, antibody fragment, or chemiluminescent probe.

19. The method of claim 15 further comprising the step of correlating the amount of fragment of substrate after the biologically significant amount of enzyme is exposed to an enzymatically effective amount of substrate for a time period sufficient to cause cleavage of the substrate into one or a plurality of fragments with a likelihood that the subject has a benign, pre-malignant, or malignant growth of the pancreas,
wherein if the amount of substrate fragment is equal to or greater than three times the amount of substrate fragment found after reactions with control samples, then the subject is diagnosed with a pre-malignant or malignant cyst of the pancreas; and
wherein if the amount of substrate is less than or equal to 1 times the amount of substrate fragment found after reactions with control samples, then the subject is diagnosed with a benign, mucinous cyst of the pancreas.

20. The method of claim 15, wherein the TPP-1 comprises at least about 70%, 75%, 80%, 85%, 90%, 91%, 92%, 93%, 94%, 95%, 96%, 97%, 98%, 99%, or 100% sequence identity to SEQ ID NO: 10 or is a variant thereof comprising at least about 70%, 75%, 80%, 85%, 90%, 91%, 92%, 93%, 94%, 95%, 96%, 97%, 98%, 99%, or 100% sequence identity to SEQ ID NO: 10.

21. The method of claim 15 further comprising detecting the biologically significant quantity of gastricsin and/or cathepsin E, or functional fragments or variants thereof, individually or collectively, in the sample.

22. The method of claim 15, wherein the sample is cystic fluid.

23.-53. (canceled)

54. A method of diagnosing a subject with pancreatic cancer, comprising:
(a) detecting a presence or quantifying a biologically significant amount of TPP1 and/or functional fragment thereof, in a sample of the subject, by contacting the sample with an enzymatically effective amount of substrate specific for TPP1 and/or functional fragment thereof; and
(b) diagnosing a subject with pancreatic cancer when the presence or quantity of TPP1 and/or functional fragment thereof is detected or quantified.

55. The method of claim 54, wherein the step of detecting a presence or quantifying an amount of TPP1 and/or functional fragment thereof is preceded by a step of obtaining the sample from the subject.

56. The method of claim 54, wherein step (a) further comprises:
calculating one or more scores based upon the presence, absence, or quantity of TPP1 and/or functional fragment thereof; and
wherein step (b) further comprises:
correlating the one or more scores to the presence, absence, or quantity of TPP1 and/or functional fragment thereof, such that, if the amount of TPP1 and/or functional fragment thereof is greater than the quantity of TPP1 and/or functional fragment thereof in a control sample; or, if the amount of TPP1 and/or functional fragment thereof is substantially equal to the quantity of TPP1 and/or functional fragment thereof in a sample taken from a subject known to have pancreatic cancer, then the subject is diagnosed as having pancreatic cancer.

57. The method of claim 54, further comprising detecting a presence or quantifying an amount of one or a combination of: gastricsin, cathepsin E, and/or CEA, or a functional fragment or variant of any of the aforementioned proteins.

58-96. (canceled)
